(19) United States
(12) Patent Application Publication
(10) Pub. No.: US 2024/0168005 A1
(43) Pub. Date: May 23, 2024

(54) DIAGNOSIS OF HEMOGLOBINOPATHIES VIA CELL MAGNETIC PROPERTIES

(71) Applicant: Ohio State Innovation Foundation, Columbus, OH (US)

(72) Inventors: Jeffrey CHALMERS, Upper Arlington, OH (US); Andre PALMER, Westerville, OH (US); Payal DESAI, Columbus, OH (US)

(21) Appl. No.: 18/281,113

(22) PCT Filed: Mar. 7, 2022
(86) PCT No.: PCT/US2022/019110

§ 371 Date: Sep. 8, 2023

Related U.S. Application Data
(60) Provisional application No. 63/158,609, filed on Mar. 9, 2021.

(51) Int. Cl. G01N 33/487; G01N 15/01; G01N 15/1031; G01N 33/49

(52) U.S. Cl. CPC G01N 33/48735; G01N 15/01; G01N 15/1031; G01N 33/49; G01N 2015/012

(57) ABSTRACT

The present disclosure provides methods to diagnose and/or treat hemoglobinopathies via comparative analysis of cell magnetic properties.

### CROSS-REFERENCE TO RELATED APPLICATIONS

This application claims the benefit of priority to U.S. Provisional Application No. 63/158,609, filed Mar. 9, 2021, the disclosure of which is incorporated herein by reference in its entirety.

### STATEMENT REGARDING FEDERALLY SPONSORED RESEARCH OR DEVELOPMENT

This invention was made with government support under HL131720 awarded by the National Institutes of Health. The Government has certain rights in the invention.

### TECHNICAL FIELD

This disclosure relates to methods of diagnosis and treatment of medical disorders, and more particularly to methods to diagnose and/or treat hemoglobinopathies.

### BACKGROUND

The United States Centers for Disease Control estimates that sickle cell disease (SCD) affects 100,000 Americans (see https://www.cdc.gov/ncbddd/sicklecell/data.html. Accessed on 6 April 2018). SCD is an inherited disease and is caused by the replacement of a single glutamic acid residue by valine in the β-chain of hemoglobin (Hb). The mutated form of Hb is called sickle cell Hb (HbS). This mutation causes HbS to aggregate into polymers inside the red blood cell (RBC) upon deoxygenation, which causes the RBC to adopt a sickle shape instead of its usual biconcave disc shape (see Kaushansky, K., Lichtman, M. A., Prchal, J., Levi, M. M., Press, O. W., Burns, L. J., and Caliguri, M. (2017) Williams Hematology, McGraw Hill, New York). Sickled RBCs have difficulty traversing capillaries and other small vessels normally, which can lead to vascular occlusion causing downstream end organ damage known as sickle cell crisis or vaso-occlusive crisis (VOC). The organs that are commonly affected include the lungs, brain, and spleen (see Novelli, E. M., and Gladwin, M. T. (2016) Crises in Sickle Cell Disease. Chest 149, 1082-1093). There is significant morbidity from VOCs that can lead to prolonged hospitalization, recurrent infections, and a shorter life expectancy.

A mainstay of treatment includes red blood cell transfusions for acute and chronic complications. Because of the large number of RBC units that SCD patients often receive to prevent and treat complications of VOCs, iron overload is a feared and very real adverse effect of transfusion that causes significant morbidity and mortality (see Coates, T. D., and Wood, J. C. (2017) How we manage iron overload in sickle cell patients. Br J Haematol 177, 703-716; Marsella, M., and Borgna-Pignatti, C. (2014) Transfusional iron overload and iron chelation therapy in thalassemia major and sickle cell disease. Hematology/oncology clinics of North America 28, 703-727, vi; and Shander, A., Cappellini, M. D., and Goodnough, L T. (2009) Iron overload and toxicity: the hidden risk of multiple blood transfusions. Vox Sang 97, 185-197). Transfusions also carry other immediate risks (acute reactions) and delayed risks, including red blood cell alloimmunization that can lead to life threatening delayed hemolytic transfusion reactions with very high mortality rates.

Although some medications such as hydroxyurea can mitigate the effect of sickling HbS (see Pule, G. D., Mowla, S., Novitzky, N., Wiysonge, C. S., and Wonkam, A. (2015), A systematic review of known mechanisms of hydroxyurea-induced fetal hemoglobin for treatment of sickle cell disease. Expert Rev Hematol 8, 669-679), transfusions plays a significant role in both preventing and treating complications of VOC's. The goal of RBC transfusion is to dilute the concentration of the SCD patient's HbS containing RBCs with normal Hb (known as HbA); containing RBCs obtained from a healthy blood donor. RBC transfusion can be performed in two ways: a simple RBC transfusion involves administering a unit from the blood bank to the patient thereby diluting their concentration of HbS containing RBCs but increasing the patient's total blood volume. A more complex transfusion strategy, known as erythrocytapheresis (or RBC exchange transfusion), involves connecting the patient to an expensive and complex machine that uses centrifugation to selectively remove the SCD patient's autologous HbS-containing RBCs and replace them with HbA containing allogenic RBCs from the blood bank (see Kelly, S., Quirolo, K., Marsh, A., Neumayr, L., Garcia, A., and Custer, B. (2016) Erythrocytapheresis for chronic transfusion therapy in sickle cell disease: survey of current practices and review of the literature. Transfusion 56, 2877-2888; Michot, J. M., Driss, F., Guitton, C., Moh Klaren, J., Lefebvre, F., Chamillard, X., Gallon, P., Fourn, E., Pela, A. M., Tertian, G., Le Bras, P., Chantalat-Auger, C., Delfraissy, J. F., Goujard, C., and Lambotte, 0. (2014) Immunohematologic tolerance of chronic transfusion exchanges with erythrocytapheresis in sickle cell disease. Transfusion; and Wahl, S. K., Garcia, A., Hagar, W., Gildengorin, G., Quirolo, K., and Vichinsky, E. (2012) Lower alloimmunization rates in pediatric sickle cell patients on chronic erythrocytapheresis compared to chronic simple transfusions. Transfusion 52, 2671-2676). RBC exchange transfusion is only available at large academic hospitals, and is thus not a treatment option for all patients without access to such a center.

In addition to vaso-occlusion, patients with SCD have increased rates of chronic hemolytic anemia leading to acute and chronic injury to the lungs, heart, kidneys, bone, and the central nervous system (see Piel, F., B., Steingber, M. H., Rees, D. C., (Apr. 20, 2017,) Sickle Cell Disease. New England Journal of Medicine 376; 16:1561-1573). Additionally, SCD patients will have episodes of acute VOC, which are flare ups of vaso-occulsion of small blood vessels in almost any organ. These vaso-occulsions are generally associated with extreme pain, and potentially localized infarction, leading to hemolysis and anemia. While some triggers for these flare ups may be avoidable, most are either unpredictable or uncontrollable, such as a seemingly unrelated weather change or an infection. Currently, there is no way to arrest an acute crisis once it starts. VOCs are managed with supportive measures such as RBC transfusion as described above, and supportive care with infusion of intravenous fluids and pain medication, frequently opiates, for palliation of symptoms. These acute symptoms are generally managed in an urgent care facility such as an emergency room, where the healthcare providers may not be familiar with the disease or patient. When patients present for care, emergency room providers must rely on patient's self-report that they are in VOC as there is currently no rapid SCD crisis detection test. Lack of available tests both hinders research on potential treatments and also accentuates health disparities and access to timely care (see Ann T. Farrell, J. P., Ankit A. Desai, Adetola A. Kassim, Jeffrey Lebensburger, Mark C. Walters, Daniel E. Bauer, Rae M. Blaylark, Donna M. DiMichele, Mark T. Gladwin, Nancy S. Green, Kathryn Hassell, Gregory J. Kato, Elizabeth S. Klings,17 Donald B. Kohn, Lakshmanan Krishnamurti, Jane Little, Julie Makani, Punam Malik, Patrick T. McGann, Caterina Minniti, Claudia R. Morris, Isaac Odame, Patricia Ann Oneal, Rosanna Setse, Poornima Sharma, and Shalini Shenoy. (2019) End points for sickle cell disease clinical trials: renal and cardiopulmonary, cure, and low-resource settings. Blood Advances 10 DECEMBER, 3 4002).

Many adult patients with SCD can describe instances of presenting for acute care during a crisis and being treated like a drug-seeking malingerer. This is because compounding the difficulty of managing a disease that is diagnosed without the benefit of a specific laboratory test or physical sign, the patients are frequently African American, and they are asking for opiates as this is the only palliative therapy. With the complex interaction of race, healthcare, and the opiate epidemic, patients with SCD have suffered from significant healthcare racism, leading to poor health outcomes and continued early mortality. They have failed to benefit from modern scientific and medical advances compared to other rare diseases (see Melillo, G. (2020) Study Finds Funding Disparities Between Sickle Cell Disease, Cystic Fibrosis).

While there are a number of treatments that attempt to manage SCD, the only potential cure is bone marrow transplantation, which carries both significant morbidity and mortality risk. While promising, genomic treatments which corrects the specific mutation to in the HbS gene are still in the early experimental stages and the toxicity as well as the potential for a durable response are still unclear. Additionally, access to either bone marrow transplant or gene therapy for the larger more affected global population may be limited by financial constraints. Thus, for the foreseeable future, SCD patients will continue to present in terrible pain and suffer organ damage during an acute crisis to emergency departments.

Thus, there is a clear need for a simple, point of care test that detects the presence of HbS-containing RB C s or other pathological abnormalities found with other hemoglobinopathies. Such diagnostic methods would relieve the burden on the patient of having to prove that they are legitimate candidates for opioid therapy, and on the doctor who has to decide if they are providing appropriate analgesia or contributing to the ongoing opioid epidemic. Such diagnostic testing would also allow for scientific advancement by allowing testing of novel drugs to treat the disease with a concrete endpoint. This present disclosure addresses these as well as other needs.

### SUMMARY

The present disclosure provides methods which are useful in the diagnosis of hemoglobinopathies based upon observed changes in the magnetic properties of cells which harbor their associated pathophysiological features. The present methods may prove useful in classifying and quantifying the level of disease state in patients with these disorders, such as the degree of pain experienced by a sickle cell disease patient. Such methods may help identify at-risk populations that would need disease modification and treatment intensification, providing the ability to scientifically prove and validate patient experience and significantly altering the patient/provider relationship in a disease fraught with health disparities.

In one aspect, a method of identifying a test cell with a pathophysiological change associated with a change in a magnetic property as compared to a standard cell is provided, the method comprising:
a. obtaining a test cell;
b. measuring the magnetic property of the test cell;
c. comparing the magnetic property of the test cell to the standard cell, where the standard cell is a normal cell without the pathophysiological change or a standardized version of a normal cell; and
d. identifying that the test cell has the pathophysiological change, wherein the test cell exhibits different magnetic properties as compared to the standard cell.

In another aspect, a method of diagnosing a subject with a hemoglobinopathy is provided, the method comprising:
a. obtaining a blood sample from the subject;
b. extracting red blood cells (RBCs) from the blood sample;
c. measuring a magnetic property of the RBCs;
d. comparing the magnetic property of the RBCs to a control, wherein the control is a normal cell showing no pathophysiological change resulting from the hemoglobinopathy or a standardized version of a normal cell;
e. detecting pathological cells amongst the RBCs, wherein said pathological cells exhibit different magnetic properties as compared to the control; and
f. diagnosing the subject with the hemoglobinopathy.

In a further aspect, a method of treating a subject with a hemoglobinopathy is provided, the method comprising:
a. obtaining a blood sample from the subject;
b. extracting red blood cells (RBCs) from the blood sample;
c. measuring a magnetic property of the RBCs;
d. comparing the magnetic property of the RBCs to a control, wherein the control sets a cutoff point which determined a need for a treatment;
e. determining that the subject is in need of the treatment; and
f. administering the treatment to the subject.

In another aspect, a kit for detecting defective red blood cells is provided, the kit comprising a CTV device and a computer system, wherein the computer system comprises software which has been programmed to detect defective blood cells.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 provides the sample preparation procedure scheme as described in the Examples. First, RBCs from whole blood samples (for HD and NTP samples) were collected by centrifugation. All RBC samples (from HD, TP, and NTP) were washed three times with PBS. The density of the samples was estimated using a Percoll gradient and density marker beads. The washed RBCs were analyzed using Coulter Counter (size/concentration distribution) and BOBS (for determining the oxygen-Hb equilibrium curve). For CTV analysis, oxyHb-RBCs were processed directly, and metHb-RBCs and deoxyHb-RBCs were obtained by treating the RBCs with NaNO₂ and Na₂S₂O₄, respectively. The same protocol was applied on all blood samples, which were processed the same day of the blood draw.

FIGS. 2A, 2B, and 2C provide scatter plot and cumulative distribution curves of um and uₛ for oxyHb-RBCs, deoxyHb-RBCs and metHb-RBCs for HD (FIG. 2A), NTP (FIG. 2B), and TP (FIG. 2C) samples as presented in Table 1 of the Examples. The dot plot shows uₘ and uₛ values of individual RBCs, whereas the middle and bottom panels present the uₘ and uₛ cumulative distribution curves for each donor/patient, respectively.

FIGS. 3A, 3B, and 3C provide histograms of magnetic (left panel) and settling (right panel) velocities for oxyHb-RBCs (FIG. 3A), deoxyHb-RBCs (FIG. 3B), and metHb-RBCs (FIG. 3C). The data contained on each panel combines all the RBCs for each group of samples (i.e., HD, TP and NTP).

FIGS. 4A and 4B provide the average MCH (FIG. 4A) and MCHC (FIG. 4B) values for HD, NTP, and TP samples when both the deoxyHb-RBC and the metHb-RBC data are employed for the calculations.

FIG. 5A provides histograms of the RBC diameter measured on the Coulter Counter and grouped for HD, TP, and NTP samples. The histograms closely overlay and there is very little difference in cell diameter between the RBC sources.

FIG. 5B provides the metHb-RBC settling velocity, which is proportional to cell diameter squared, for HD, TP, and NTP donors. The slight right shift in uₛ suggests a difference in cell density between healthy and SCD patients, as present in Table 1 of the Examples.

FIG. 6 provides oxygen equilibrium curves for HC, NTP, and TP RBC samples averaged and overlaid in a scatter plot. HD exhibit the highest oxygen affinity, followed by TP and lastly NTP.

FIG. 7 provides the theoretical magnetic moment of HD (solid), TP (dashed), and NTP (dotted) RBC samples as a function of pO₂. The lower oxygenation affinity of NTP samples in comparison to HD and TP results in a lower saturation, and therefore higher magnetic moments under intermediate oxygen levels.

FIGS. 8A-8C show the principle of CTV measurement (FIG. 8A) and RBC velocity scatter plots with marginal histograms, RBC sedimentation against magnetophoresis at pO₂=0.1 mmHg (FIG. 8B) and at pO₂=160 mmHg (FIG. 8C). Notably, there is an effect of pO₂ on RBC magnetophoresis but not on RBC sedimentation, and the magnitudes of the two quantities is comparable.

FIG. 9 provides a comparison of the quantity of Hb per RBC between CTM measurements and UV-visible spectrophotometry. The UV-visible spectrophotometry approach required cell lysis and 3 replicate dilutions and measurements of total Hb concentration. This value was further divided by the RBC count to obtain a per cell value. In contrast, the CTV value is based on the mean and standard deviation of >1,000 individually tracked cells.

FIGS. 10A-10C show a scatter plot of settling versus magnetic velocity and volume versus pgHb/cell (FIG. 10A), a histogram of magnetic velocity versus Hb concentration (FIG. 10B), and a histogram of settling velocity (FIG. 10C).

FIGS. 11A-11C show three representative results of the CTV analysis for normal blood (FIG. 11A), pure SCD blood (FIG. 11B), and transfusion waste (FIG. 11C).

FIG. 12 shows histograms of the magnetic velocity of oxyRBCs from a normal donor (top), non-transfused SCD donor (middle), and RBCs from an apheresis transfusion waste bad. The vertical dotted line represents a threshold of magnetic velocity of 1×10⁻⁴ mm/s.

FIG. 13 shows the correlation of the percent of RBC population with a magnetic velocity higher than 10⁻⁴ mm/s as a function of donor/patient pain category. n corresponds to sample size.

FIG. 14 is the corrected version of FIG. 13 using equation 8.

FIG. 15 shows examples of Percol separation of normal RBCs, apheresis transfusion waste, non-transfused, category 1 SCD patient RBCs, and calibration beads, from left to right. The bottom portion provides a calibration graph to relate position to density of the calibration beads.

FIGS. 16A-16D show CTV, BOBs, and Coulter Counter analysis of Percol fractionated, non-transfused, SD patient blood that has a category 1 pain/disease state classification. The density fractions are coded as following: F0, density fractionated blood, F1-F4, lightest to heaviest fraction. FIG. 16A is air saturated, oxyRBCs and FIG. 16B is deoxygenated deoxyRBCs. FIG. 16C is BOBs data for each of the fractions, and FIG. 16D is cell diameter measurements from the Coulter Counter.

Like reference symbols in the various drawings indicate like elements.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the to filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a cell”, “a magnetic property”, or “a hemoglobinopathy”, includes, but is not limited to, two or more such cells, magnetic properties, or hemoglobinopathies, and the like.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a hemoglobinopathy. The effect can be therapeutic in terms of a partial or complete cure of a disease, condition, symptom or adverse effect attributed to the disease, disorder, or condition. The term “treatment” as used herein can include any treatment of a disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

In one aspect, a method of identifying a test cell with a pathophysiological change associated with a change in a magnetic property as compared to a standard cell is provided, the method comprising:
a. obtaining a test cell;
b. measuring the magnetic property of the test cell;
c. comparing the magnetic property of the test cell to the standard cell, where the standard cell is a normal cell without the pathophysiological change or a standardized version of a normal cell; and
d. identifying that the test cell has the pathophysiological change, wherein the test cell exhibits different magnetic properties as compared to the standard cell.

The test cell may be any cell in which a pathophysiological change has been determined to be associated with a change in a magnetic property for the cell. In some embodiments, the test cell is paramagnetic. In some embodiments, the standard cell is a normal cell of the same type as the test cell but without the presence of the tested pathophysiological change. In some embodiments, the standard cell is a standardized version of a normal cell. In some embodiments, the standard cell is diamagnetic. The cells may be obtained from a subject (i.e., a human or animal), a cell culture, or a bioreactor.

In some embodiments, the test cell and/or standard cell is a blood cell. In some embodiments, the test cell and/or standard cell is selected from a monocyte, a lymphocyte, a neutrophil, an eosinophil, a basophil, a macrophage, a platelet, or an erythrocyte (i.e., a red blood cell). In some embodiments, the test cell and/or standard cell is a monocyte. In some embodiments, the test cell and/or standard cell is a lymphocyte. In some embodiments, the test cell and/or standard cell is a neutrophil. In some embodiments, the test cell and/or standard cell is a basophil. In some embodiments, the test cell and/or standard cell is a macrophage. In some embodiments, the test cell and/or standard cell is a platelet. In some embodiments, the test cell and/or standard cell is a red blood cell. In some embodiments, the test cell is a sickle red blood cell.

The magnetic properties of hemoglobin (Hb), and by extensions, red blood cells (RBCs) were studied by the pioneering work of Linus Pauling in 1935 (see Pauling, L. (1935) The Oxygen Equilibrium of Hemoglobin and Its Structural Interpretation. Proceedings of the National Academy of Sciences 21, 186-191; and Pauling, L., and Coryell, C. D. (1936) The Magnetic Properties and Structure of Hemoglobin, Oxyhemoglobin and Carbonmonoxyhemoglobin. Proc Natl Acad Sci U S A 22, 210-216) and led to research on magnetic cell separation (see Melville, D. (1975) Direct magnetic separation of red cells from whole blood. Nature 255, 706; and Paul, F., Roath, S., and Melville, D. (1978) Differential blood cell separation using a high gradient magnetic field. British journal of haematology 38, 273-280) and application to functional nuclear magnetic resonance imaging (fMRI) (see Fabry, M. E., and San George, R. C. (1983) Effect of magnetic susceptibility on nuclear magnetic resonance signals arising from red cells: a warning. Biochemistry 22, 4119-4125). The process of interconversion between the diamagnetic (oxygenated) and paramagnetic (deoxygenated) forms of Hb is directly related to the essential function of Hb, which facilitates oxygen transport between the lungs and peripheral tissues. The changes in magnetic susceptibility of Hb-O₂ species in the range from 0-100% oxygen saturation are large enough to be detectable by RBC motion analysis in strong applied magnetic fields (see Okazaki, M., Maeda, N., and Shiga, T. (1986) Drift of an erythrocyte flow line due to the magnetic field. Experientia 42, 842-843; Zborowski, M., Ostera, G. R., Moore, L. R., Milliron, S., Chalmers, J. J., and Schechter, A. N. (2003) Red blood cell magnetophoresis. Biophys J 84, 2638-2645; and Kim, J., Gomez-Pastora, J., Gilbert, C. J., Weigand, M., Walters, N. A., Reategui, E., Palmer, A. F., Yazer, M., Zborowski, M., and Chalmers, J. J. (2020) Quantification of the Mean and Distribution of Hemoglobin Content in Normal Human Blood Using Cell Tracking Velocimetry. Anal Chem 92, 1956-1962).

In some embodiments, the pathophysiological change is associated with a hemoglobinopathy. As used herein, the term “hemoglobinopathy” includes any disorder involving the presence of an abnormal hemoglobin molecule in the blood. Examples of hemoglobinopathies include, but are not limited to, hemoglobin C disease, hemoglobin sickle cell disease (SCD), sickle cell anemia, and thalassemias. Also included are hemoglobinopathies in which a combination of abnormal hemoglobins are present in the blood (e.g., sickle cell/Hb-C disease). In some embodiments, if the test cell has been obtained from a subject, the subject has been diagnosed with a hemoglobinopathy.

Non-limiting exemplary hemoglobinopathies include sickle cell disease (including, but not limited to, homozygous for hemoglobin S and a variety of sickle cell syndromes that result from inheritance of the sickle cell gene in compound heterozygosity with other mutant beta globin genes, including, but not limited to, hemoglobin SC disease (HbSC), sickle beta(+) thalassemia, sickle beta(0) thalassemia, sickle apha thalassemia, sickle delta beta (0) thalassemia, sickle Hb Lepore, sickle HbD, sickle HbO Arab, and sickle HbE), β-thalassemia (including, but not limited to, β-thalassemia major (also known as Cooley's anemia) and β-thalassemia intermedia, and hemoglobin H disease (α-thalassemia with α⁺-α⁰ phenotype). Non-limiting exemplary genetic mutations that cause sickle cell disease include Hb SS, which is hemoglobin with an E6V mutation and one β chain with a β121 Glu→Gln mutation; sickle-HbO Arab, which is hemoglobin with one β chain with an E6V mutation and one β chain with a β121(GH4)gGlu→Lys mutation; and Hb SE, which is hemoglobin with one β chain with an E6V mutation and one β chain with an E26K mutation. Non-limiting exemplary genetic mutations that cause β-thalassemia include various R-mutations, such as IVS II-I, CD 36/37, CD 41/42, CD 39; IVS1-6; IVS1-110, CD71/72, IVS1-5, IVS1-1, cd26, ivs2-654, cap+1, cd19, -28, -29 IVS1-2, InCD (T-G) and CD17; and rare β-mutations, i.e., InCD (A-C), CD8/9, CD43, -86, CD15, Poly A, Poly TIC, IVS2-1, CD1, CD35/36, CD27/28, CD16, CD37, and 619bpDEL. Non-limiting exemplary genetic mutations that cause Hb H disease include α⁺-α⁰ phenotypes such as α2 Poly A (AATAAA→AATA—), α2 Poly A (AATAAA→AATGAA), and α2 Poly A (AATAAA→AATGAA), and α2 Poly A (AATAAA→AATAAG); α⁺ phenotypes such as α2 CD 142 (TAA→CAA), α2 CD 142 (TAA→AAA), and α2 CD 142 (TAA→TAT), and α⁰ phenotypes such as −α^{3.7} Init CD (ATG→GTG), -^{SEA}, -^{THAI}, -^{MED II}, -^{BRIT}, -^{MED I}, -^{SA}, -(α)^{20.5}, and -^{FIL}.

Hemoglobinopathies comprise inherited blood disorders or diseases that primarily affect red blood cells. Hemoglobinopathies typically affect either the structure or production of the hemoglobin molecule. Hemoglobin is a tetramer composed of two α-globin and two non-α-globin chains working in conjunction with heme to transport oxygen in the blood. Normal adult hemoglobin (HbA) is designated α^{A}₂β^{A}₂. Variant hemoglobin is derived from gene abnormalities affecting the α-globin genes (HBA1 or HBA2) or β-globin (HBB) structural genes (exons). More than a thousand hemoglobin variants have been identified relative to changes in the globin chains. Qualitative changes correspond to amino acid substitutions resulting in hemoglobinopathies. Quantitative changes like amino acid insertions, deletions or mutations in the intervening sequences (introns) correspond to thalassemia and result in decreased globin chain production.

Alpha thalassemias are caused by changes (deletions, point mutations, insertions, etc.) in the α-globin genes. Production of a-globin is controlled by the four alleles of HBA1 and HBA2. In the deletional type of α-thalassemias, the number of α-globin gene deletions correlates to disease severity. One α-globin gene deletion is unremarkable (also called a silent carrier) whereas a two α-globin gene deletion (α-thalassemia trait) and three α-globin gene deletion (HbH disease) have varied clinical and hematological features. A four α-globin gene deletion (Hb Bart's Hydrops fetalis) is severe and not typically compatible with life.

Beta globin variants more commonly seen include HbS, HbC, HbD, HbE and HbG. A mutation in one β-globin subunit results in a combination of variant and normal hemoglobin and denotes carrier or trait status, also known as the heterozygous state. Mutations in both β-globin subunits result in disease based on a homozygous or heterozygous expression. In the case of sickle cell anemia (HbSS), mutations are homozygous with production of HbS. Other disease classed under sickle cell disease (SCD), for example Hb SE, Hb SC and HbS β-thalassemia, are heterozygous expressions. Regardless of an α-globin or β-globin variant, severity of disease can range from insignificant to serious or life threatening.

As used herein, the term “sickle cell disease” refers to a group of autosomal recessive genetic blood disorders, which results from mutations in a globin gene and which is characterized by red blood cells that assume an abnormal, rigid, sickle shape. They are defined by the present of β^{S}-gene coding for a β-globin chain variant in which glutamic acid is substituted by valine at amino acid position 6 of the peptide, and second β-gene that has a mutation that allows for the crystallization of HbS leading to a clinical phenotype. As used herein, the term “sickle cell anemia” refers to a specific form of sickle cell disease in patients who are homozygous for the mutation that causes HbS. Other common forms of sickle cell disease include HbS/β-thalassemia, HbS/HbC, and HbS/HbD.

As used herein, the term “thalassemia” refers to a hereditary disorder characterized by defective production of hemoglobin. Examples of thalassemias include α- and β-thalassemia. B-thalassemias are caused by a mutation in the beta globin chain and can occur in a major or minor form. In the major form of β-thalassemia, children are normal at birth, but develop anemia during the first year of life. The mild form of β-thalassemia produces small red blood cells and the thalassemias are caused by deletion of a gene or genes from the globin chain. Alpha-thalassemia typically results from deletions involving the HBA1 and HBA2 genes. Both of these genes encode α-globin, which is a subunit of hemoglobin. There are two copies of the HBA1 gene and two copies of the HBA2 gene in each cellular genome. As a result, there are four alleles that produce α-globin. The different types of α-thalassemia result from the loss of some or all of these alleles. Hb Bart syndrome, the most severe form of α-thalassemia, results from the loss of all four α-globin alleles. HbH disease is caused by a loss of three of the four α-globin alleles. In these two conditions, a shortage of α-globin prevents cells from making normal hemoglobin. Instead, cells produce abnormal forms of hemoglobin called hemoglobin Bart (Hb Bart) or hemoglobin H (HbH). These abnormal hemoglobin molecules cannot effectively carry oxygen to the body's tissues. The substitution of Hb Bart of HbH for normal hemoglobin causes anemia and the other serious health problems associated with α-thalassemia.

In some embodiments, the hemoglobinopathy may be selected from the group consisting of: hemoglobin C disease, hemoglobin sickle cell disease (SCD), sickle cell anemia, hereditary anemia, thalassemia, β-thalassemia, thalassemia major, thalassemia intermedia, 60 -thalassemia, and hemoglobin H disease. In some embodiments, the hemoglobinopathy is β-thalassemia. In some embodiments, the hemoglobinopathy is sickle cell anemia.

In some embodiments, the standard cell used for comparison is from a subject without the hemoglobinopathy. In some embodiments, the pathophysiological change comprises a reduced or increased level of hemoglobin (Hb), iron, or other paramagnetic atom in the test cell. In other embodiments, the pathophysiological change is not the result of a change in the level of hemoglobin and instead results from a change in the physical properties of hemoglobin as a result of one or more mutations.

The magnetic property of the test cell and/or standard cell may be measured using any suitable method as would be known in the art. In some embodiments, the magnetic property of the cell is measured using a cell tracking velocity (CTV) device, magnetic deposition, or magnetic flow field fractionation. In some embodiments, the magnetic property of the cell is measured using a CTV device.

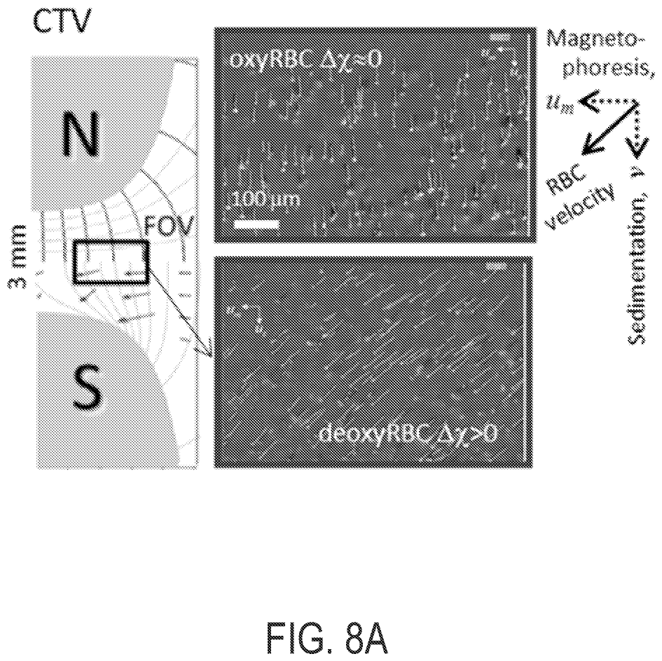

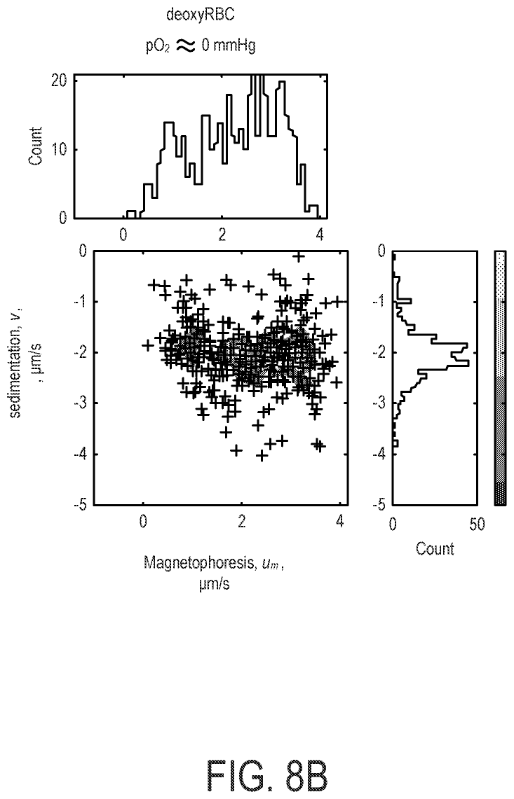

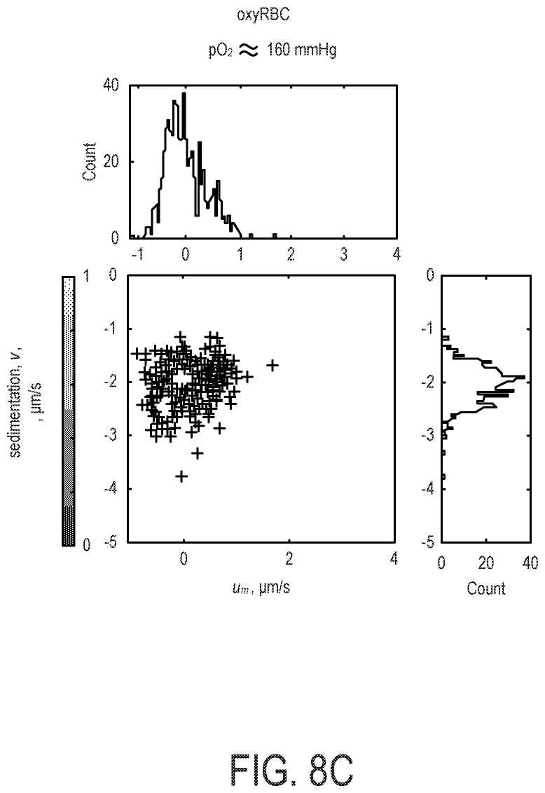

Magnetic field-induced RBC motion in viscous media is referred to as “magnetophoresis” (Zborowski, M., and Chalmers, J. J. (1999) Magnetophoresis: Fundamentals and applications. Wiley Encyclopedia of Electrical and Electronics Engineering, 1-23) and has been extensively characterized using the technique of cell tracking velocimetry (see McCloskey, K. E., Chalmers, J. J., and Zborowski, M. (2003) Magnetic cell separation: characterization of magnetophoretic mobility. Anal Chem 75, 6868-6874; and Zborowski, M., Sun, L., Moore, L. R., and Chalmers, J. J. (1999) Rapid cell isolation by magnetic flow sorting for applications in tissue engineering. ASAIO journal 45, 127-130). The CTV device allows accurate measurements of the magnetophoretic and sedimentation components of the RBC velocity vector, repeated on a sample of up to a few thousand cells. Typically, magnetic velocity is observed along the horizontal magnetic gradient and sedimentation velocity is observed along the vertical gravitational acceleration direction (see FIGS. 8A-8C). CTV is orders of magnitude more sensitive than other established methods, such as superconducting quantum interference device-magnetic properties measurement system (SQUID-MPMS), which measures bulk magnetic properties, but not the magnetic properties of individual cells (see Xue, W., Moore, L. R., Nakano, N., Chalmers, J. J., and Zborowski, M. (2019) Single cell magnetometry by magnetophoresis vs. bulk cell suspension by SQUI-MPMS:—A comparison. Journal of Magnetism and Magnetic Materials 474).

In typical embodiments, the CTV device comprises a microscope, a camera, and a magnet. The magnet may comprise a permanent magnet, a superconducting magnet, or an electromagnet. In particular embodiments, the magnet comprises NdFeB magnets. Microfluidic channels are used to track the movement of the cell within the CTV device.

In some embodiments, the CTV device measures magnetically induced velocity (uₘ) and/or gravity induced settling velocity (uₛ) of the cells. In some embodiments, the CTV device may further measure cell density. Typically, the CTV device creates a magnetic energy gradient (Sₘ) which is perpendicular to gravity. The magnetically induced horizontal and vertical velocities of the cells are then measured.

In some embodiments, uₘ and uₛ are determined as follows:

$${u}_{m} = \frac{( {χ}_{Cell} - {χ}_{Fluid} ) {V}_{Cell}}{{f}_{d} 3 π {D}_{Cell} η} {S}_{m} \qquad ( 1 )$$

$${u}_{s} = \frac{( {ρ}_{Cell} - {ρ}_{Fluid} ) {V}_{Cell}}{{f}_{d} 3 π {D}_{Cell} η} g \qquad ( 2 )$$

where the subscripts cell and fluid refer to the cell and the suspending fluid, χ is the magnetic susceptibility, ρ is the density, D and V are the diameter and volume of the cell, η is the viscosity of the suspending fluid, f_{d} is the drag coefficient, and g is the acceleration due to gravity (i.e., 9.8 m/s²). Typically, f_{d} is 1.0 for spheres and 1.23 for disc-shaped cells (e.g., erythrocytes).

In some embodiments, Sₘ is defined by:

$${S}_{m} = \frac{\vert ∇ {B}^{2} \vert}{2 {μ}_{0}} \qquad ( 3 )$$

where μ₀ is the permeability of free space and B is the magnetic flux density at the cell.

Rearranging equations 1 and 2 provides the following as a measure of the magnetic susceptibility of the cell:

$${χ}_{Cell} = {χ}_{Fluid} + \frac{{u}_{m}}{{u}_{s}} \frac{( {ρ}_{Cell} - {ρ}_{Fluid} ) g}{{S}_{m}} \qquad ( 4 )$$

The magnetic susceptibility of the cell is a material property of its constituents (for example, hemoglobin) and does not depend upon the volume, diameter, or fluid viscosity of the cell.

In some embodiments, the magnetic and settling velocity is associated with the mass and concentration of hemoglobin (Hb) in the cell. In particular, the mean corpuscular Hb (MCH) and mean corpuscular Hb concentration (MCHC) is about:

$$M C H = \frac{9 * {2}^{0.5} π}{{S}_{m} * ( {χ}_{m , Hb} + {χ}_{m , globin} - {χ}_{{H}_{2} O} ) * {V}_{Hb}} * [ \text{} \frac{( {f}_{d} * {u}_{m} ) * {( {f}_{d} * {u}_{s} )}^{0.5} * {η}^{1.5}}{{Δρ}^{0.5} * {g}^{0.5}} ] * {10}^{3} * {MW}_{Hb} \qquad ( 5 )$$

$$M C H C = \frac{( \frac{{u}_{m}}{{u}_{s}} ) ( Δρ ) ( \frac{g}{{s}_{m}} )}{( {χ}_{m , Hb} + {χ}_{m , globin} - {χ}_{{H}_{2} O} ) * {V}_{Hb}} * {MW}_{Hb} \qquad ( 6 )$$

where V_{Hb} is the molar volume of Hb (i.e., 48.23 L/mol), MW_{Hb} is the molecular weight of Hb (i.e., 64,450 g/mol), χ_{m,globin} is the molar susceptibility of a globin chain of Hb (i.e., 37,830×10⁻⁹ L/mol), and χ_{H2O} is the molar susceptibility of water (i.e., −12.97×10⁻⁹ L/mol).

In some embodiments, images of the cell's location in the CTV device is captured using an imaging system. This imaging system may be used to calculate uₘ and uₛ. MCH, MCHC, and χ_{RBC} may each be calculated using a computer.

In another aspect, a method of diagnosing a subject with a hemoglobinopathy is provided, the method comprising:
a. obtaining a blood sample from the subject;
b. extracting red blood cells from the blood sample;
c. measuring a magnetic property of the red blood cells;
d. comparing the magnetic property of the red blood cells to a control, wherein the control is a normal cell showing no pathophysiological change resulting from the hemoglobinopathy or a standardized version of a normal cell;
e. detecting pathological cells amongst the red blood cells, wherein said pathological cells exhibit different magnetic properties as compared to the control; and
f. diagnosing the subject with the hemoglobinopathy.

In some embodiments, the hemoglobinopathy comprises sickle cell disease, sickle cell anemia, hereditary anemia, thalassemia, beta-thalassemia, thalassemia major, thalassemia intermedia, alpha-thalassemia, or hemoglobin H disease. In particular embodiments, the hemoglobinopathy comprises sickle cell disease.

In another aspect, a method of treating a subject with a hemoglobinopathy is provided, the method comprising:
a. obtaining a blood sample from the subject;
b. extracting red blood cells from the blood sample;
c. measuring a magnetic property of the red blood cells;
d. comparing the magnetic property of the red blood cells to a control, wherein the control sets a cutoff point which determined a need for a treatment;
e. determining that the subject is in need of the treatment; and
f. administering the treatment to the subject.

In some embodiments, the hemoglobinopathy comprises sickle cell disease, sickle cell anemia, hereditary anemia, thalassemia, beta-thalassemia, thalassemia major, thalassemia intermedia, alpha-thalassemia, or hemoglobin H disease. In particular embodiments, the hemoglobinopathy comprises sickle cell disease. The treatment to be administered will vary depending on the particular hemoglobinopathy to be treated.

For sickle cell disease, treatment involves a number of measures. Management of sickle cell disease is usually aimed at avoiding pain episodes, relieving symptoms and preventing complications. Hydroxyurea/hydroxycarbamide reduces the frequency of painful crises and might reduce the need for blood transfusions and hospitalization. L-glutamine oral powder may help reduce the frequency of pain crises. Crizanlizumab reduces the frequency of pain crises. Narcotics or non-steroidal anti-inflammatory drugs may be administered to relieve pain during sickle cell pain crises. Voxelotor may reduce blood sickling in people with sickle cell disease. In some embodiments, the treatment for sickle cell disease may comprise a blood transfusion. In a red blood cell transfusion, normal red blood cells are removed from a supply of donated blood then given through a vein to the subj ect.

Treatments for sickle cell disease may be for amelioration of any number of crises which may occur in such patients. The terms “sickle cell crisis” or “sickling crisis” may be used to describe several independent acute conditions occurring in patients with sickle cell disease, which results in anemia and crises that could be of many types, including the vaso-occlusive crisis, aplastic crisis, splenic sequestration crisis, hemolytic crisis, and others. Most episodes of sickle cell crises last between five and seven days. Even though infection, dehydration, and acidosis can act as triggers, in most instances no predisposing cause is identified.

The vaso-occlusive crisis is caused by sickle-shaped red blood cells that obstruct capillaries and restrict blood flow to an organ, resulting in ischemia, pain, necrosis, and often organ damage. The frequency, severity, and duration of these crises vary considerably. Painful crises are treated with hydration, analgesics, and blood transfusion; pain management requires opioid drug administration at regular intervals until the crisis has settles. For milder crises, a subgroup of patients manages on NSAIDs such as diclofenac or naproxen. For more severe crises, most patients require inpatient management for intravenous opioids; patient-controlled analgesia devices are commonly used in this setting. Vaso-occlusive crisis involving organs such as the penis or lungs are considered an emergency and treated with red blood cell transfusions. Incentive spirometry, a technique to encourage deep breathing to minimize the development of atelectasis, is often recommended.

The spleen is frequently affected in sickle cell disease, as the sickle-shaped red blood cells causes narrowing of blood vessels and reduced function in clearing defective cells. It is usually infarcted before the end of childhood. This spleen damage increases the risk of infection from encapsulated organisms. Splenic sequestration crises are acute, painful enlargements of the spleen, caused by intrasplenic trapping of red cells and resulting in a precipitous fall in hemoglobin levels with the potential for hypovolemic shock. Sequestration crises are considered an emergency. If not treated, patients may die within 1-2 hours due to circulatory failure. Management is supportive, sometimes with blood transfusion. These crises are transient; they continue for 3-4 hours and may last for one day.

Acute chest syndrome is defined by at least two of the following signs or symptoms: chest pain, fever, pulmonary infiltrate or focal abnormality, respiratory symptoms, or hypoxemia. It is the second most common complication and accounts for about 25% of deaths in patients with sickle cell disease. Most cases present with vaso-occlusive crises and then develop acute chest syndrome. Management is similar to vaso-occlusive crisis, with the addition of antibiotics (usually a quinolone or macrolide, since cell wall-deficient bacteria are thought to contribute to the syndrome), oxygen supplementation for hypoxia, and close observation. Should the pulmonary infiltrate worsen or oxygen requirements increase, simple blood transfusion or exchange transfusion is indicated. The latter involves the exchange of a significant portion of the person's red cell mass for normal red cells, which decreases the level of hemoglobin S in the patient's blood.

Aplastic crises are acute worsening's of the patient's baseline anemia, producing pale appearance, fast heart rate, and fatigue. This crises is normally trigger by parvovirus B19, which directly affects production of red blood cells by invading the red cell precursors and multiplying in and destroying them. Parvovirus infection almost completely prevent red blood cell production for two to three days. The shorted red blood cell half life of sickle cell disease patients results in an abrupt, life-threatening situation. Reticulocyte counts drop dramatically during the disease, and the rapid turnover of red blood cells leads to a drop in hemoglobin. This crisis takes 4 to 7 days to disappear, with most patients being managed supportively (but some needing transfusions).

Hemolytic crises are acute drops in hemoglobin level resulting from red blood cells breaking down at a faster rate. This is particularly common in people with coexistent G6PD deficiency. Management is supportive, but sometimes blood transfusions are necessary.

Treatment for alpha-thalassemia may include blood transfusions to maintain hemoglobin at a level that reduces the symptoms of anemia. The decision to initiate transfusions depends on the clinical severity of the disease. Further treatments of alpha-thalassemia may include daily doses of folic acid, splenectomy, and iron chelation therapy. Beta-thalassemia may be treated by blood transfusions, iron chelation therapy, daily folic acid, and bone marrow transplant.

In another aspect, a kit is provided for detecting defective red blood cells, the kit comprising a CTV device and a computer system, wherein the computer system comprises software which has been programmed to detect defective blood cells. In some embodiments, to the kit further comprises a component for collecting a blood sample, for example a needle. In some embodiments, the kit further comprises a sample input component which can be placed in the CTV device. In some embodiments, the sample input component comprises a channel. In some embodiments, the software is programmed to detect whether the subject has a hemoglobinopathy. In some embodiments, the software is programmed to detect whether a subject with a hemoglobinopathy is in need of treatment. In some embodiments, the computer system is a handheld device, such as a smart device, for example a phone, watch or tablet.

The following further embodiments of the invention are also provided. Embodiment 1. A method of identifying a test cell with a pathophysiological change associated with a change in a magnetic property as compared to a standard cell, the method comprising:
a. obtaining a test cell;
b. measuring the magnetic property of the test cell;
c. comparing the magnetic property of the test cell to the standard cell, where the standard cell is a normal cell without the pathophysiological change or a standardized version of a normal cell; and
d. identifying that the test cell has the pathophysiological change, wherein the test cell exhibits different magnetic properties as compared to the standard cell.

Embodiment 2. The method of embodiment 1, wherein the cell is selected from a monocyte, a lymphocyte, a neutrophil, an eosinophil, a basophil, a macrophage, a platelet, or an erythrocyte (red blood cell).

Embodiment 3. The method of embodiment 1 or 2, wherein the cell is an erythrocyte (red blood cell).

Embodiment 4. The method of embodiment 3, wherein the test cell is a sickle red blood cell.

Embodiment 5. The method of any one of embodiments 1-4, wherein the pathophysiological change is associated with a hemoglobinopathy.

Embodiment 6. The method of embodiment 5, wherein the hemoglobinopathy comprises sickle cell disease (SCD), sickle cell anemia, sickle cell trait, hereditary anemia, thalassemia, β-thalassemia, thalassemia major, thalassemia intermedia, α-thalassemia, or hemoglobin H disease.

Embodiment 7. The method of embodiment 5, wherein the hemoglobinopathy comprises sickle cell disease.

Embodiment 8. The method of any one of embodiments 5-7, wherein the standard cell used for comparison is from a subject without the hemoglobinopathy.

Embodiment 9. The method of any one of embodiments 1-8, wherein the pathophysiological change comprises a reduced or increased level of hemoglobin (Hb), iron, or other paramagnetic atom.

Embodiment 10. The method of any one of embodiments 1-9, wherein the test cell is paramagnetic.

Embodiment 11. The method of any one of embodiments 1-10, wherein the magnetic property of the cell is determined using a cell tracking velocity (CTV) device, magnetic deposition, or magnetic flow field fractionation.

Embodiment 12. The method of embodiment 11, wherein the magnetic property of the cell is determined using a CTV device.

Embodiment 13. The method of embodiment 12, wherein a microscope, camera, and a magnet are used as part of the CTV device.

Embodiment 14. The method of embodiment 13, wherein the magnet comprises a permanent magnet, a superconducting magnet, or an electromagnet.

Embodiment 15. The method of embodiments 13 or 14, wherein the magnet comprises NdFeB magnets.

Embodiment 16. The method of any one of embodiments 12-15, wherein microfluidic channels are used to track the movement of the test cell.

Embodiment 17. The method of any one of embodiments 12-16, wherein the CTV device measures magnetically induced velocity (uₘ) of the cells, gravity induced settling velocity (uₛ) of the cells, or cell density.

Embodiment 18. The method of any one of embodiments 12-17, wherein the CTV device creates a magnetic energy gradient (Sₘ) which is perpendicular to gravity.

Embodiment 19. The method of embodiment 18, wherein magnetically induced horizontal and vertical velocities of the cells are measured.

Embodiment 20. The method of embodiment 19, wherein uₘ and uₛ are as follows:

$$\begin{matrix}{u}_{m} = \frac{{χ}_{Cell} - {χ}_{Fluid} ) {V}_{Cell}}{{f}_{d} 3 π {D}_{Cell} η} {S}_{m} & ( 1 ) \\ {u}_{s} = \frac{( {ρ}_{Cell} - {ρ}_{FLuid} ) {V}_{Cell}}{{f}_{d} 3 π {D}_{Cell} η} g & ( 2 )\end{matrix}$$

where the subscripts cell and fluid refer to the cell and the suspending fluid, χ is the magnetic susceptibility, ρ is the density, D and V are the diameter and volume of the cell, η is the viscosity of the suspending fluid, f_{d} is the drag coefficient, and g is the acceleration due to gravity,
and where Sₘ is defined by:

$${S}_{m} = \frac{\vert ∇ {B}^{2} \vert}{2 {μ}_{0}} \qquad ( 3 )$$

where μ₀ is the permeability of free space and B is the magnetic flux density at the cell.

Embodiment 21. The method of embodiment 19, wherein an additive is included to modify the density of the suspending fluid.

Embodiment 22. The method of embodiment 21, Wherein the density of the suspending fluid differs from a density of a typical cell buffer.

Embodiment 23. The method of embodiment 19, wherein an additive is included to modify the magnetic susceptibility of the suspending fluid.

Embodiment 24. The method of embodiment 23, wherein the magnetic susceptibility of the suspending fluid differs from a magnetic susceptibility of a typical cell buffer.

Embodiment 25. The method of embodiment 20, wherein rearranging Equations (1) and. (2) leads to:

$${χ}_{Cell} = {χ}_{Fluid} + \frac{{u}_{m}}{{u}_{s}} \frac{( {ρ}_{Cell} - {ρ}_{Fluid} ) g}{{S}_{m}} \qquad ( 4 )$$

Embodiment 26. The method of embodiment 25, wherein the magnetic susceptibility of the cell is a material property of its constituents and does not depend on volume, diameter, or fluid viscosity of the cell.

Embodiment 27. The method of embodiment 26, wherein the relationship between magnetic and settling velocity and the mass and concentration of hemoglobin (Hb) in the cell, the mean corpuscular Hb (MCH) and the mean corpuscular Hb concentration (MCHC) is about:

$$MCH = \frac{9 * {2}^{0.5} π}{{S}_{m} * ( {χ}_{m , Hb} + {χ}_{m , globin} - {χ}_{{H}_{2} O} ) * {V}_{Hb}} * [ \frac{( {f}_{d} * {u}_{m} ) * {( {f}_{d} * {u}_{s} )}^{0.5} * {η}^{1.5}}{{Δρ}^{0.5} * {g}^{0.5}} ] * {10}^{3} * {MW}_{Hb} \qquad ( 5 )$$

$$MCHC = \frac{( \frac{{u}_{m}}{{u}_{s}} ) ( Δρ ) ( \frac{g}{{S}_{m}} )}{( {χ}_{m , Hb} + {χ}_{m , globin} - {χ}_{{H}_{2} O} ) * {V}_{Hb}} * {MW}_{Hb} \qquad ( 6 )$$

where V_{Hb} is the molar volume of Hb, MW_{Hb} is the molecular weight of Hb, χ_{m,globin} is the molar susceptibility of a globin chain of Hb, and χ_{H2O} is the molar susceptibility of water.

Embodiment 28. The method of any one of embodiments 12-27, wherein images of the cell's location is captured using an imaging system.

Embodiment 29. The method of embodiment 28, where inthe imaging system is used to calculate uₘ and uₛ.

Embodiment 30. The method of embodiment 29, wherein MCH and MCHC are calculated by computer.

Embodiment 31. The method of any one of embodiments 1-30, wherein multiple cells are analyzed in parallel.

Embodiment 32. The method of embodiment 31, wherein the cells are obtained from a human subject, an animal subject, a cell culture, or a bioreactor.

Embodiment 33. The method of embodiment 32, wherein the subject has been diagnosed with a hemoglobinopathy.

Embodiment 34. The method of embodiment 33, wherein the hemoglobinopathy comprises sickle cell disease.

Embodiment 35. A method of diagnosing a subject with a hemoglobinopathy, the method comprising:
a. obtaining a blood sample from the subject;
b. extracting red blood cells from the blood sample;
c. measuring a magnetic property of the red blood cells;
d. comparing the magnetic property of the red blood cells to a control, wherein the control is a normal cell showing no pathophysiological change resulting from the hemoglobinopathy or a standardized version of a normal cell;
e. detecting pathological cells amongst the red blood cells, wherein said pathological cells exhibit different magnetic properties as compared to the control; and
f. diagnosing the subject with the hemoglobinopathy.

Embodiment 36. The method of embodiment 35, wherein the hemoglobinopathy comprises sickle cell disease (SCD), sickle cell anemia, sickle cell trait, hereditary anemia, thalassemia, β-thalassemia, thalassemia major, thalassemia intermedia, α-thalassemia, or hemoglobin H disease.

Embodiment 37. The method of embodiment 35, wherein the hemoglobinopathy comprises sickle cell disease.

Embodiment 38. The method of any one of embodiments 35-37, wherein the pathological cells are more or less paramagnetic than the control.

Embodiment 39. The method of any one of embodiments 35-38, wherein the magnetic to property of the cell is determined using a cell tracking velocity (CTV) device, magnetic deposition, or magnetic flow field fractionation.

Embodiment 40. The method of embodiment 39, wherein the magnetic property of the cell is determined using a CTV device.

Embodiment 41. The method of embodiment 40, wherein a microscope, camera, and a magnet are used as part of the CTV device.

Embodiment 42. The method of embodiment 41, wherein the magnet comprises a permanent magnet, a superconducting magnet, or an electromagnet.

Embodiment 43. The method of embodiments 41 or 42, wherein the magnet comprises NdFeB magnets.

Embodiment 44. The method of any one of embodiments 40-43, wherein microfluidic channels are used to track the movement of the test cell.

Embodiment 45. The method of any one of embodiments 40-44, wherein the CTV device measures magnetically induced velocity (uₘ) of the cells, gravity induced settling velocity (uₛ) of the cells, or cell density.

Embodiment 46. The method of any one of embodiments 40-45, wherein the CTV device creates a magnetic energy gradient (Sₘ) which is perpendicular to gravity.

Embodiment 47. The method of embodiment 46, wherein magnetically induced horizontal and vertical velocities of the cells are measured.

Embodiment 48. The method of embodiment 47, wherein uₘ and uₛ are as follows:

$$\begin{matrix}{u}_{m} = \frac{{χ}_{Cell} - {χ}_{Fluid} ) {V}_{Cell}}{{f}_{d} 3 π {D}_{Cell} η} {S}_{m} & ( 1 ) \\ {u}_{s} = \frac{( {ρ}_{Cell} - {ρ}_{FLuid} ) {V}_{Cell}}{{f}_{d} 3 π {D}_{Cell} η} g & ( 2 )\end{matrix}$$

where the subscripts cell and fluid refer to the cell and the suspending fluid, χ is the magnetic susceptibility, ρ is the density, D and V are the diameter and volume of the cell, η is the viscosity of the suspending fluid, f_{d} is the drag coefficient, and g is the acceleration due to gravity,
and where Sₘ is defined by:

$${S}_{m} = \frac{\vert ∇ {B}^{2} \vert}{2 {μ}_{0}} \qquad ( 3 )$$

where μ₀ is the permeability of free space and B is the magnetic flux density at the cell.

Embodiment 49. The method of embodiment 48, wherein an additive is included to modify the density of the suspending fluid.

Embodiment 50. The method of embodiment 49, wherein the density of the suspending fluid differs from a density of a typical cell buffer.

Embodiment 51. The method of embodiment 48, wherein an additive is included to modify the magnetic susceptibility of the suspending fluid.

Embodiment 52. The method of embodiment 51, wherein the magnetic susceptibility of the suspending fluid differs from a magnetic susceptibility of a typical cell buffer.

Embodiment 53. The method of embodiment 48, wherein rearranging Equations (1) and (2) leads to:

$${χ}_{Cell} = {χ}_{Fluid} + \frac{{u}_{m}}{{u}_{s}} \frac{( {ρ}_{Cell} - {ρ}_{Fluid} ) g}{{S}_{m}} \qquad ( 4 )$$

Embodiment 54. The method of embodiment 53, wherein the magnetic susceptibility of the cell is a material property of its constituents and does not depend on volume, diameter, or fluid viscosity of the cell.

Embodiment 55. The method of embodiment 54, wherein the relationship between magnetic and settling velocity and the mass and concentration of hemoglobin (Hb) in the cell, the mean corpuscular (MCH) and the mean corpuscular Hb concentration (MCHC) is about:

$$MCH = \frac{9 * {2}^{0.5} π}{{S}_{m} * ( {χ}_{m , Hb} + {χ}_{m , globin} - {χ}_{{H}_{2} O} ) * {V}_{Hb}} * [ \frac{( {f}_{d} * {u}_{m} ) * {( {f}_{d} * {u}_{s} )}^{0.5} * {η}^{1.5}}{{Δρ}^{0.5} * {g}^{0.5}} ] * {10}^{3} * {MW}_{Hb} \qquad ( 5 )$$

$$MCHC = \frac{( \frac{{u}_{m}}{{u}_{s}} ) ( Δρ ) ( \frac{g}{{S}_{m}} )}{( {χ}_{m , Hb} + {χ}_{m , globin} - {χ}_{{H}_{2} O} ) * {V}_{Hb}} * {MW}_{Hb} \qquad ( 6 )$$

where V_{Hb} is the molar volume of Hb, MW_{Hb} is the molecular weight of Hb, χ_{m,globin} is the molar susceptibility of a globin chain of Hb, and χ_{H2O} is the molar susceptibility of water.

Embodiment 56. The method of any one of embodiments 40-55, wherein images of the cell's location is captured using an imaging system.

Embodiment 57. The method of embodiment 56, wherein the imaging system is used to calculate uₘ and uₛ.

Embodiment 58. The method of embodiment 57, wherein MCH and MCHC are calculated by computer.

Embodiment 59. The method of any one of embodiments 35-58, wherein multiple cells are analyzed in parallel.

Embodiment 60. A method of treating a subject with a hemoglobinopathy, the method comprising:
a. obtaining a blood sample from the subject;
b. extracting red blood cells from the blood sample;
c. measuring a magnetic property of the red blood cells;
d. comparing the magnetic property of the red blood cells to a control, wherein the control sets a cutoff point which determined a need for a treatment;
e. determining that the subject is in need of the treatment; and
f. administering the treatment to the subject.

Embodiment 61. The method of embodiment 60, wherein the hemoglobinopathy comprises sickle cell disease (SCD), sickle cell anemia, sickle cell trait, hereditary anemia, thalassemia, β-thalassemia, thalassemia major, thalassemia intermedia, α-thalassemia, or hemoglobin H disease.

Embodiment 62. The method of embodiment 60, wherein the hemoglobinopathy comprises sickle cell disease.

Embodiment 63. The method of any one of embodiments 60-62, wherein the treatment comprises pain management.

Embodiment 64. The method of any one of embodiments 60-63, wherein the treatment comprises blood transfusion.

Embodiment 65. The method of any one of embodiments 60-64, wherein the treatment comprises administration of a therapeutic agent for the hemoglobinopathy.

Embodiment 66. The method of embodiment 65, wherein the therapeutic agent comprises hydroxycarbamide, crizanlizumab, voxelotor, luspatercept, or combinations thereof.

Embodiment 67. The method of any one of embodiments 60-66, wherein the treatment comprises removal of pathological red blood cells from the subject.

Embodiment 68. The method of any one of embodiments 60-67, wherein the magnetic property of the cell is determined using a cell tracking velocity (CTV) device, magnetic deposition, or magnetic flow field fractionation.

Embodiment 69. The method of embodiment 68, wherein the magnetic property of the cell is determined using a CTV device.

Embodiment 70. The method of embodiment 69, wherein a microscope, camera, and a magnet are used as part of the CTV device.

Embodiment 71. The method of embodiment 70, wherein the magnet comprises a permanent magnet, a superconducting magnet, or an electromagnet.

Embodiment 72. The method of embodiments 70 or 71, wherein the magnet comprises NdFeB magnets.

Embodiment 73. The method of any one of embodiments 69-72, wherein microfluidic channels are used to track the movement of the test cell.

Embodiment 74. The method of any one of embodiments 69-73, wherein the CTV device measures magnetically induced velocity (uₘ) of the cells, gravity induced settling velocity (uₛ) of the cells, or cell density.

Embodiment 75. The method of any one of embodiments 69-74, wherein the CTV device creates a magnetic energy gradient (Sₘ) which is perpendicular to gravity.

Embodiment 76. The method of embodiment 75, wherein magnetically induced horizontal and vertical velocities of the cells are measured.

Embodiment 77. The method of embodiment 76, wherein uₘ and uₛ are as follows:

$$\begin{matrix}{u}_{m} = \frac{{χ}_{Cell} - {χ}_{Fluid} ) {V}_{Cell}}{{f}_{d} 3 π {D}_{Cell} η} {S}_{m} & ( 1 ) \\ {u}_{s} = \frac{( {ρ}_{Cell} - {ρ}_{FLuid} ) {V}_{Cell}}{{f}_{d} 3 π {D}_{Cell} η} g & ( 2 )\end{matrix}$$

where the subscripts cell and fluid refer to the cell and the suspending fluid, χ is the magnetic susceptibility, ρ is the density, D and V are the diameter and volume of the cell, η is the viscosity of the suspending fluid, f_{d} is the drag coefficient, and g is the acceleration due to gravity,
and where Sₘ is defined by:

$${S}_{m} = \frac{\vert ∇ {B}^{2} \vert}{2 {μ}_{0}} \qquad ( 3 )$$

where μ₀ is the permeability of free space and B is the magnetic flux density at the cell.

Embodiment 78. The method of embodiment 77, wherein an additive is included to modify the density of the suspending fluid.

Embodiment 79. The method of embodiment 78, wherein the density of the suspending fluid differs from a density of a typical cell buffer.

Embodiment 80. The method of embodiment 77, wherein an additive is included to modify the magnetic susceptibility of the suspending fluid.

Embodiment 81. The method of embodiment 80, wherein the magnetic susceptibility of the suspending fluid differs from a magnetic susceptibility of a typical cell buffer.

Embodiment 82. The method of embodiment 77, wherein rearranging Equations (1) and (2) leads to:

$${χ}_{Cell} = {χ}_{Fluid} + \frac{{u}_{m}}{{u}_{s}} \frac{( {ρ}_{Cell} - {ρ}_{Fluid} ) g}{{S}_{m}} \qquad ( 4 )$$

Embodiment 83. The method of embodiment 82, wherein the magnetic susceptibility of the cell is a material property of its constituents and does not depend on volume, diameter, or fluid viscosity of the cell.

Embodiment 84. The method of embodiment 83, wherein the relationship between magnetic and settling velocity and the mass and concentration of hemoglobin (Hb) in the cell, the mean corpuscular Hb (MCH) and the mean corpuscular Hb concentration (MCHC) is about:

$$MCH = \frac{9 * {2}^{0.5} π}{{S}_{m} * ( {χ}_{m , Hb} + {χ}_{m , globin} - {χ}_{{H}_{2} O} ) * {V}_{Hb}} * [ \frac{( {f}_{d} * {u}_{m} ) * {( {f}_{d} * {u}_{s} )}^{0.5} * {η}^{1.5}}{{Δρ}^{0.5} * {g}^{0.5}} ] * {10}^{3} * {MW}_{Hb} \qquad ( 5 )$$

$$MCHC = \frac{( \frac{{u}_{m}}{{u}_{s}} ) ( Δρ ) ( \frac{g}{{S}_{m}} )}{( {χ}_{m , Hb} + {χ}_{m , globin} - {χ}_{{H}_{2} O} ) * {V}_{Hb}} * {MW}_{Hb} \qquad ( 6 )$$

where V_{Hb} is the molar volume of Hb, MW_{Hb} is the molecular weight of Hb, χ_{m,globin} is the molar susceptibility of a globin chain of Hb, and χ_{H2O} is the molar susceptibility of water.

Embodiment 85. The method of any one of embodiments 69-84, wherein images of the cell's location is captured using an imaging system.

Embodiment 86. The method of embodiment 85, wherein the imaging system is used to calculate uₘ and uₛ.

Embodiment 87. The method of embodiment 86, wherein MCH and MCHC are calculated by computer.

Embodiment 88. The method of any one of embodiments 60-87, wherein multiple cells are analyzed in parallel.

Embodiment 89. A kit for detecting defective red blood cells, the kit comprising a CTV device and a computer system, wherein the computer system comprises software which has been programmed to detect defective blood cells.

Embodiment 90. The kit of embodiment 89, further comprising a component for collecting a blood sample.

Embodiment 91. The kit of embodiment 90, wherein the component for collecting a blood sample comprises a needle.

Embodiment 92. The kit of any one of embodiments 89-91, further comprising a sample input component which can be placed in the CTV device.

Embodiment 93. The kit of embodiment 92, wherein said sample input component comprises a channel.

Embodiment 94. The kit of any one of embodiments 89-93, wherein the software is programmed to detect whether the subject has a hemoglobinopathy.

Embodiment 95. The kit of any one of embodiments 89-93, wherein the software is programmed to detect whether a subject with a hemoglobinopathy is in need of treatment.

Embodiment 96. The kit of any one of embodiments 89-95, wherein the computer system is a handheld device.

Embodiment 97. The kit of embodiment 96, wherein the handheld device is a smart device.

Embodiment 98. The kit of embodiment 97, wherein the smart device is a phone, watch or tablet.

Embodiment 99. The kit of any one of embodiments 89-98, further comprising a suspension fluid into which the cells are suspended.

Embodiment 100. The kit of embodiment 99, wherein the suspension fluid has a density which differs from a density of a typical cell buffer.

Embodiment 101. The kit of embodiment 99 or 100, wherein the suspension fluid has a magnetic susceptibility which differs from a magnetic susceptibility of a typical cell buffer.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are put forth so as to provide those of ordinary skill in the art with a complete disclosure and description of how the compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

### The Unique Magnetic Signature of Sickle Red Blood Cells: A Comparison Between the Red Blook Cells of Transfused and Non-Transfused Sickle Cell Disease Patients and Healthy Donors

Sickle cell disease (SCD) is an inherited blood disorder that affects millions of people worldwide, especially in low-resource regions of the world, where a rapid and affordable test to properly diagnose the disease would be highly valued. A technique that could be used to simultaneously analyze, quantify and potentially separate the patient's sickle RBCs from healthy RBCs is magnetophoresis, but the magnetic characteristics of sickle RBCs have yet to be reported. In this example, we present the single cell magnetic characterization of RBCs obtained from SCD patients. Sufficient single cells are analyzed, from patient samples undergoing transfusion therapy and not yet having transfusion therapy (TP and NTP, respectively), such that mean and distributions of the mobility of these single RBCs are created in the form of histograms which facilitated comparisons to RBCs similarly analyzed from healthy donors (HD). The magnetic characterization is obtained using an instrument referred to as Cell Tracking Velocimetry (CTV) that quantitatively characterizes the RBC response to magnetic and gravitational fields. The magnetic properties of RBCs containing oxygenated, deoxygenated hemoglobin (Hb) and methemoglobin (oxyHb-RBCs, deoxyHb-RBCs and metHb-RBCs) are further determined. The SCD samples report the highest magnetic character, especially when they are compared to oxyHb-RBCs from HD, which implies their impaired oxygen binding capabilities. Also, the oxygen-Hb equilibrium curves are obtained to estimate the magnetic character of the cells under intermediate oxygen levels. Our results confirm the higher magnetic moment of SCD blood (NTP) under intermediate oxygen levels. These data demonstrate the potential feasibility of magnetophoresis to identify, quantify and separate sickle RBCs from healthy RBCs.

### Introduction

Sickle cell disease (SCD) is an inherited blood disorder that affects millions of people worldwide; it affects 25% of people living in Central and West Africa and approximately 100,000 people in the United States, mostly of African descent (see American Red Cross—Sickle Cell Disease. Accessed via: https://www.redcrossblood.org/donate-blood/blood-types/diversity/sicklecell.html on 06/19/2021; R. E. Ware, M. de Montalembert, L. Tshilolo, M. R. Abboud. Sickle cell disease. The Lancet 390 (2017) 311-323; and S. M. Knowlton, I. Sencan, Y. Aytar, J. Khoory, M. M. Heeney, I. C. Ghiran, S. Tasoglu. Sickle cell detection using a smartphone. Scientific Reports 5 (2015) 15022) have the most serious form, the so called homozygous, HbSS, form of the disease. This hemoglobinopathy is the first described instance of a “molecular disease” and is caused by a single amino acid mutation in the β-globin gene of hemoglobin (Hb). Upon deoxygenation, sickle Hb (HbS) enters the tense (T) conformational state, where the mutant valine is able to induce polymerization of HbS, which is subsequently reported to dehydrate and shrivel the erythrocyte. This results in hardened and elongated red blood cells (RBCs) that do not uptake oxygen efficiently, obstruct blood vessels, and increase blood viscosity (see W. A. Eaton, E. R. Henry, J. Hofrichter, A. Mozzarelli. Is cooperative oxygen binding by hemoglobin really understood? Nature Structural Biology 6 (1999) 351-358). In addition to the previously mentioned, homozygous HbSS, other severe forms of SCD include compound heterozygous conditions, such as HbC with HbS (HbSC), HbS with β-thalassaemia (HbS/β⁰-thalassaemia or HbS/β⁺-thalassaemia), and HbS with other beta-globin variants such as HbSD or HbSO_{Arab}, all of which express sufficient HbS to cause intracellular sickling. The inheritance of both HbA and HbS (HbAS) corresponds to sickle cell trait; strictly not a form of SCD but that may be associated with adverse health outcomes. Sickle cell trait affects between 1 and 3 million Americans, 8 to 10% of African Americans, and more than 100 million people worldwide (see American Society of Hematology—Sickle Cell Trait. Accessed via: https://www.hematology.org/education/patients/anemia/sickle-cell-trait on 09/09/2021).

The RBCs in a person without SCD circulate in the bloodstream for approximately 120 days and are replaced by new cells synthesized in the bone marrow; however, it is reported that sickle RBCs survive only 10 to 20 days in the circulation, resulting in hemolytic anemia characterized by a decrease in the number of circulating RBCs and total [Hb]. Sickle cells are stiff, distorted in shape and sometimes block small blood vessels, causing vaso-occlusive crises (VOCs). Individuals with SCD suffer a range of conditions, including acute anemia, infections, tissue and organ damage, severe pain, acute chest syndrome, and strokes. The median life expectancy for those with SCD is 40 to 50 years. Although gene therapy approaches have been successful, there is no widely used cure for SCD. In some cases, hydroxyurea is prescribed to increase the levels of Hb and fetal Hb (HbF) and to reduce the frequency of painful episodes (see R. K. Agrawal, R. K. Patel, V. Shah, L. Nainiwal, B. Trivedi. Hydroxyurea in Sickle Cell Disease: Drug Review. Indian Journal of Hematology and Blood Transfusion 30 (2014) 91-96). Another common treatment is regular blood transfusions. However, the annual cost of transfusions rises to several thousand dollars per year, and although they are effective, they are financially inaccessible to the majority of SCD patients (see P. Boma Muteb, J. F. J. Kaluila Mamba, P. Muhau Pfutila, V. Bilo, J. D. Panda Mulefu, D. A. Diallo. Effectiveness, safety, and cost of partial exchange transfusions in patients with sickle-cell anemia at a sickle cell disease center in sub-Saharan Africa. Medecine et Sante Tropicales 27 (2017) 387-391). Moreover, they pose significant clinical challenges and transfusion-related complications exist, such as alloimmunization, delayed hemolytic transfusion reactions, and iron overload (see S. T. Chou, M. Alsawas, R. M. Fasano, J. J. Field, J. E. Hendrickson, J. Howard, M. Kameka, J. L. Kwiatkowski, F. Pirenne, P. A. Shi, S. R. Stowell, S. L. Thein, C. M. Westhoff, T. E. Wong, E. A. Akl. American Society of Hematology 2020 guidelines for sickle cell disease: transfusion support. Blood Advances 4 (2020) 327-355; G. Stussi, A. Buser, A. Holbro. Red Blood Cells: Exchange, Transfuse, or Deplete. Transfusion Medicine and Hemotherapy 46 (2019) 407-416; and R. L. Saylors, B. Watkins, S. Saccente, X. Tang. Comparison of Automated Red Cell Exchange Transfusion and Simple Transfusion for the Treatment of Children with Sickle Cell Disease Acute Chest Syndrome. Pediatric Blood & Cancer 60 (2013) 1952-1956).

SCD is best managed when diagnosed early, but since SCD is most prevalent in low-resource regions of the world, screening for SCD is rare and diagnosis at the point-of-care (POC) is challenging. In fact, in many regions, most affected children die undiagnosed before the age of 5 years (see C. Steele, A. Sinski, J. Asibey, M. D. Hardy-Dessources, G. Elana, C. Brennan, I. Odame, C. Hoppe, M. Geisberg, E. Serrao, C. T. Quinn. Point-of-care screening for sickle cell disease in low-resource settings: A multi-center evaluation of HemoTypeSC, a novel rapid test. American Journal of Hematology 94 (2019) 39-45). Thus, a rapid and affordable test for SCD is needed. A technique that could be used to simultaneously analyze, quantify, and potentially separate the patient's sickle RBCs from healthy RBCs is magnetophoresis. The magnetic susceptibility of Hb depends on the oxidative state and oxygen binding of Hb's four iron (Fe) atoms. The works of Pauling, Coryell, and others indicated that for deoxygenated ferrous Hb and for methemoglobin (the oxidized form of Hb), the presence of unpaired electrons when the Fe is attached to the surrounding protoporphyrin and histidine side chain by ionic bonds makes these species paramagnetic whereas oxygenated, ferrous Hb has no unpaired electrons due to its covalent bonds and is diamagnetic in nature (L. Pauling, C. Coryell. The magnetic properties and structure of the hemochromogens and related substances. Proceedings of the National Academy of Sciences of the United States of America 22 (1936) 159-163; Pauling, C. Coryell. The magnetic properties and structure of hemoglobin, oxyhemoglobin and carbonmonoxyhemoglobin. Proceedings of the National Academy of Sciences of the United States of America 22 (1936) 210-216; and M. Zborowski, G. R. Ostera, L. R. Moore, S. Milliron, J. J. Chalmers, A. N. Schechter. Red Blood Cell Magnetophoresis. Biophysical Journal 84 (2003) 2638-2645). The separation and analysis of RBCs from other cell types using magnetic means has been reported widely in the literature (see D. Melville, F. Paul, and S. Roath. Direct magnetic separation of red cells from whole blood. Nature 255 (1975) 706; J. Gomez-Pastora, X. Xue, I. H. Karampelas, E. Bringas, E. P. Furlani, I. Ortiz. Analysis of separators for magnetic beads recovery: From large systems to multifunctional microdevices. Separation and Purification Technology 172 (2017) 16-31; J. Kim, J. Gomez-Pastora, C. J. Gilbert, M. Weigand, N. A. Walters, E. Reategui, A. F. Palmer, M. Yazer, M. Zborowski, J. J. Chalmers. Quantification of the Mean and Distribution of Hemoglobin Content in Normal Human Blood Using Cell Tracking Velocimetry. Analytical Chemistry 92 (2020) 1956-1962; J. Kim, J. Gomez-Pastora, M. Weigand, M. Potgieter, N. A. Walters, E. Reategui, A. F. Palmer, M. Yazer, M. Zborowski, J. J. Chalmers. A Subpopulation of Monocytes in Normal Human Blood Has Significant Magnetic Susceptibility: Quantification and Potential Implications. Cytometry Part A 95 (2019) 478-487; and E. P. Furlani. Magnetophoretic separation of blood cells at the microscale. Journal of Physics D: Applied Physics 40 (2007) 1313). Moreover, magnetic devices (magnetic levitation devices, mostly) have been proposed for the diagnosis of SCD (see S. M. Knowlton, B. Yenilmez, R. Amin, S. Tasoglu. Magnetic Levitation Coupled with Portable Imaging and Analysis for Disease Diagnostics. Journal of Visualized Experiments 120 (2017) e55012; L. Thompson, B. Pinckney, S. Lu, M. Gregory, J. Tigges, I. Ghiran. Quantification of Cellular Densities and Antigenic Properties using Magnetic Levitation. Journal of Visualized Experiments 171 (2021) e62550; M. Baday, S. Calamak, N. G. Durmus, R. W. Davis, L. M. Steinmetz, U. Demirci. Integrating Cell Phone Imaging with Magnetic Levitation (i-LEV) for Label-Free Blood Analysis at the Point-of-Living. Small 12 (2016) 1222-1229; A. A. Kumar, M. R. Patton, J. W. Hennek, S. Y. R. Lee, G. D′Alesio-Spina, X. Yang, J. Kanter, S. S. Shevkoplyas, C. Brugnara, G. M. Whitesides. Density-based separation in multiphase systems provides a simple method to identify sickle cell disease. Proceedings of the National Academy of Sciences of the United States of America 111 (2014) 14864-14869; and S. Ge, A. Nemiroski, K. A. Mirica, C. R. Mace, J. W. Hennek, A. A. Kumar, G. M. Whitesides. Magnetic Levitation in Chemistry, Materials Science, and Biochemistry. Angewandte Chemie International Edition 59 (2020) 17810-17855). Nevertheless, the potential of this technique to analyze, quantify and separate sickle RBCs from SCD patients is not yet clear, since information regarding the RBC properties of SCD patients at the single cell level is not available. Especially important is understanding the physicochemical properties of Hb and how they influence the magnetic behavior of RBCs (e.g. ability to bind oxygen), along with other properties that might affect the magnetic separation, such as the density, shape and deformability of erythrocytes. It has been demonstrated that in SCD, the presence of sickle RBCs, characterized by cellular dehydration and polymerization of HbS, affects the volume, density, size, intracellular Hb concentration, and oxygen affinity of the RBC (see T. Maruyama, M. Fukata, T. Fujino. Physiological and pathophysiological significance of erythrocyte senescence, density and deformability: Important but unnoticed trinity. Journal of Biorheology 34 (2020) 61-70; C. Brugnara and N. Mohandas. Red cell indices in classification and treatment of anemias: from M. M. Wintrobes's original 1934 classification to the third millennium. Current Opinion in Hematology 20 (2013) 222-230; M. Seakins, W. N. Gibbs, P. F. Milner, J. F. Bertles. Erythrocyte Hb-S concentration. An important factor in the low oxygen affinity of blood in sickle cell anemia. The Journal of Clinical Investigation 52 (1973) 422-432; and W. A. Eaton, H. F. Bunn. Treating sickle cell disease by targeting HbS polymerization. Blood 129 (2017) 2719-2726). A recent study on bulk volume susceptibility difference between deoxyhemoglobin and oxyhemoglobin for HbS and HbA by blood-oxygen level dependent (BOLD) nuclear magnetic resonance imaging (MRI) reports no difference although, interestingly, that study shows greater spread of data for HbS than for HbA samples (see C. Eldeniz, M. M. Binkley, M. Fields, K. Guilliams, D. K. Ragan, Y. Chen, J. M. Lee, A. L. Ford, H. An. Bulk volume susceptibility difference between deoxyhemoglobin and oxyhemoglobin for HbA and HbS: A comparative study. Magnetic Resonance in Medicine 85 (2021) 3383-3393). A comparative study using ab-initio quantum-mechanical simulations of the electronic structure of HbS and HbA's active centers reports no difference in the intensity of magnetization of the Fe atom in HbS and HbA molecules not bound to 02 and a small excess of magnetization in the 02-HbS complex compared to 02-HbA complex (see D. Y. Novoselov, D. M. Korotin, V. I. Anisimov. Features of the Electronic Structure of the Active Center of an HbS Molecule. Russian Journal of Physical Chemistry A 90 (2016) 113-116). A recent, detailed quantum-mechanical simulations study on the effective magnetization of Fe in the heme cluster points to a significant contribution of the neighboring atoms of the globin molecule (see S. Mayda, Z. Kandemir, N. Bulut, S. Maekawa. Magnetic mechanism for the biological functioning of hemoglobin. Scientific Reports 10 (2020) 8569), which could be affected by the Hb polymerization in the HbS molecule.

In this example, we describe single cell scale magnetic characterization of RBCs obtained from SCD patients both requiring and not requiring chronic transfusion therapy. The analysis is carried out by using different instruments. First, an instrument referred to as Cell Tracking Velocimetry (CTV) is employed, which has been previously used to characterize the magnetic behavior of individual cells or particles (see J. Gomez-Pastora, J. Kim, V. Multanen, M. Weigand, N. A. Walters, E. Reategui, A. F. Palmer, M. H. Yazer, M. Zborowski, J. J. Chalmers. Intrinsically magnetic susceptibility in human blood and its potential impact on cell separation: Non-classical and intermediate monocytes have the strongest magnetic behavior in fresh human blood. Experimental Hematology 99 (2021) 21-31; M. R. H. Weigand, J. Gomez-Pastora, J. Kim, M. T. Kurek, R. J. Hickey, D. C. Irwin, P. W. Buehler, M. Zborowski, A. F. Palmer, J. J. Chalmers. Magnetophoretic and Spectral Characterization of Oxyhemoglobin and Deoxyhemoglobin: Chemical versus Enzymatic Processes. PLOS ONE; J. J. Chalmers, X. Jin, A. F. Palmer, M. H. Yazer, L. Moore, P. Amaya, K. Park, X. Pan, M. Zborowski. Femtogram Resolution of Iron Content on a Per Cell Basis: Ex Vivo Storage of Human Red Blood Cells Leads to Loss of Hemoglobin. Analytical Chemistry 89 (2017) 3702-3709; and W. Xue, L. R. Moore, N. Nakano, J. J. Chalmers, M. Zborowski. Single cell magnetometry by magnetophoresis vs. bulk cell suspension magnetometry by SQUID-MPMS—a comparison. Journal of Magnetism and Magnetic Materials 474 (2019) 152-160). More specifically, the CTV was used to evaluate the magnetic properties of individual RBCs containing either fully oxygenated Hb (oxyHb-RBCs), deoxygenated Hb (deoxyHb-RBCs) and methemoglobin (metHb-RBCs). Complementary to the CTV measurements, the oxygen-Hb equilibrium curves from these same samples are obtained by using a Blood Oxygen Binding System (BOBS). In addition to the oxygen-Hb equilibrium curves, estimates are made of the magnetic character of SCD samples under intermediate oxygen levels. The data obtained from SCD patient RBCs are also compared to the magnetic properties of RBCs obtained from healthy donors. Our results confirm a difference in the properties of the RBCs obtained from SCD patients and healthy donors, especially under intermediate oxygen saturation levels.

### Experimental

### Sample Preparation

A total of 15 RBC samples obtained from healthy individuals and SCD patients receiving and not receiving chronic transfusion therapy (at least for the last 3 months prior to sample collection) were collected following informed consent according to approved protocols. All experiments were performed in accordance with relevant guidelines and regulations. For the healthy donor RBC samples (HD samples), approximately 5 mL of whole blood was drawn into a 10 mL collection tube containing EDTA anticoagulant. For the SCD patients not requiring transfusion therapy (NTP samples), the same protocol was observed, where 5 mL of whole blood was collected into 10 mL tubes containing EDTA anticoagulant. For SCD patients requiring transfusion therapy (TP samples), the discarded RBCs were collected in a bag containing citrate while the patient received RBC exchange apheresis. The samples (variable volumes of the discarded RBC) were taken directly from the apheresis collection bag after the exchange was complete.

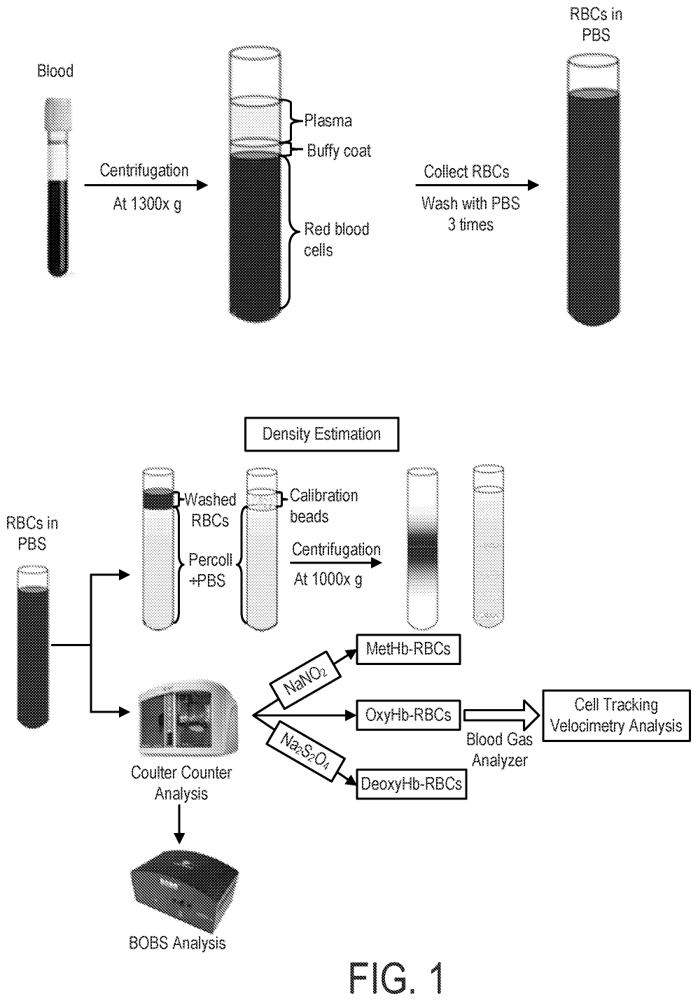

The RBCs from HD and NTP whole blood and TP apheresis waste product were washed in phosphate buffered saline (PBS) using centrifugation (three times at 1300×g for 5 minutes), as presented in FIG. 1. After washing, the average density of RBCs was determined by centrifugation (1000×g for 15 minutes) using a Percoll gradient (Cytiva Sweden AB, Sweden) and density marker beads (Amersham Biosciences AB, Sweden), which are colored microspheres of known mass density that are used for determining the density of cells in gradient columns, as shown in FIG. 1. All samples were introduced into an automated cell counter, B23005 Multisizer 4e Coulter Counter (CC, Beckman Coulter, CA), to measure the cell concentration as well as volume (and equivalent diameter) distributions.

After preparing RBC samples and determining size and concentration distributions, RBCs were divided into three aliquots designated “oxyHb-RBCs”, “deoxyHb-RBCs” and “metHb-RBCs”, for CTV analysis. OxyHb-RBCs were left open to room air for 10 min to ensure that the cells were in oxyHb state. The paramagnetic forms of RBCs (deoxyHb-RBCs and metHb-RBCs) were obtained after treating the washed RBCs with sodium dithionite (deoxyHb-RBCs) and sodium nitrite (metHb-RBCs) as previously reported in the literature (see J. Kim, M. Weigand, A. F. Palmer, M. Zborowski, M. H. Yazer, J. J. Chalmers. Single cell analysis of aged RBCs: quantitative analysis of the aged cells and byproducts. Analyst 144 (2019) 935-942). The oxygenation state of the oxyHb-RBC and deoxyHb-RBC samples was ensured by measuring the pO₂ (partial pressure of oxygen, which reflects the amount of oxygen gas dissolved in the blood sample) using a blood gas analyzer (RAPIDLab 248, Siemens Healthcare Diagnostics, German) before CTV analysis. Finally, oxyHb-RBCs were analyzed by BOBS. In FIG. 1, the workflow of the sample preparation procedure is schematized.

### Cell Tracking Velocimetry (CTV) Analysis

Once the samples were prepared, the magnetic characterization of fully oxygenated, deoxygenated and oxidized RBCs was performed using CTV. CTV uses a microscope, camera, permanent NdFeB magnets and a microfluidic channel to track the movement of cells and particles under the direct influence of magnetic and gravitational fields. More specifically, CTV measures the magnetically induced velocity (uₘ) and gravity induced settling velocity (uₛ) of cells in a region of interest, where a high, uniform, and well characterized magnetic energy gradient (Sₘ), perpendicular to gravity, is created. Once the RBCs are injected, horizontal and vertical velocities are measured, which can be related to specific cellular and magnetic field properties. The magnetically and gravitationally induced velocities, uₘ and uₛ, can be described as follows:

$$\begin{matrix}{u}_{m} = \frac{{χ}_{Cell} - {χ}_{Fluid} ) {V}_{Cell}}{{f}_{d} 3 π {D}_{Cell} η} {S}_{m} & ( 1 ) \\ {u}_{s} = \frac{( {ρ}_{Cell} - {ρ}_{FLuid} ) {V}_{Cell}}{{f}_{d} 3 π {D}_{Cell} η} g & ( 2 )\end{matrix}$$

where the subscripts cell and fluid refer to the cell and the suspending fluid, χ is the magnetic susceptibility, ρ is the density, D and V are the diameter and volume of the RBC, η is the viscosity of the suspending fluid, f_{d} is the drag coefficient (1.0 for spheres and 1.23 for disc-shaped erythrocytes) and g is the acceleration due to gravity (9.8 m/s²). Sₘ is defined by:

$${S}_{m} = \frac{\vert ∇ {B}^{2} \vert}{2 {μ}_{0}} \qquad ( 3 )$$

where μ₀ and B are the permeability of free space and the magnetic flux density at the RBC, respectively. Rearranging Equations (1) and (2) leads to:

$${χ}_{Cell} = {χ}_{Fluid} + \frac{{u}_{m}}{{u}_{s}} \frac{( {ρ}_{Cell} - {ρ}_{Fluid} ) g}{{S}_{m}} \qquad ( 4 )$$

The RBC magnetic susceptibility is the material property of its constituents and does not depend on the RBC size (volume and diameter) nor the fluid viscosity. Moreover, the relationship between the magnetic and settling velocity and the mass and concentration of Hb in the RBCS, the mean corpuscular Hb (MCH) and the mean corpuscular Hb concentration (MCHC), can be estimated as:

$$MCH = \frac{9 * {2}^{0.5} π}{{S}_{m} * ( {χ}_{m , Hb} + {χ}_{m , globin} - {χ}_{{H}_{2} O} ) * {V}_{Hb}} * [ \frac{( {f}_{d} * {u}_{m} ) * {( {f}_{d} * {u}_{s} )}^{0.5} * {η}^{1.5}}{{Δρ}^{0.5} * {g}^{0.5}} ] * {10}^{3} * {MW}_{Hb} \qquad ( 5 )$$

$$MCHC = \frac{( \frac{{u}_{m}}{{u}_{s}} ) ( Δρ ) ( \frac{g}{{S}_{m}} )}{( {χ}_{m , Hb} + {χ}_{m , globin} - {χ}_{{H}_{2} O} ) ( {V}_{Hb}} * {MW}_{Hb} \qquad ( 6 )$$

where V_{Hb}=48.23 L/mol is the molar volume of Hb, MW_{Hb} is the molecular weight of Hb (64,450 g/mol), χ_{m,globin}=−37,830×10⁻⁹ L/mol is the molar susceptibility of the globin chain, and χ_{H}_{₂}_{O}=−12.97×10⁻⁹ L/mol is the molar susceptibility of water. The molar susceptibility of the deoxygenated Hb heme group is χ_{m,deoxyHb}=50,890×10⁻⁹ L/mol, and that of the metHb heme group is χ_{m,metHb}=56,000×10⁻⁹ L/mol (all in CGS system of units). The molar susceptibility of the oxyHb heme group is zero.

After introducing the different RBC samples into the CTV, images of the cells' location were captured using an in-house program and analyzed using the Trackmate plugin on ImageJ (see J. Y. Tinevez, N. Perry, J. Schindelin, G. M. Hoopes, G. D. Reynolds, E. Laplantine, S. Y. Bednarek, S. L. Shorte, K. W. Eliceiri. TrackMate: An open and extensible platform for single-particle tracking. Methods 115 (2017) 80-90) and in-house programs to calculate uₘ and uₛ. With these data, χ_{RBC} as well as the MCH and MCHC were calculated.

### Blood Oxygen Binding System (BOBS) Measurements

Washed, oxyHb-RBCs were also analyzed in the BOBS (Loligo Systems, Viborg, Denmark). BOBS continuously performs UV-visible spectroscopy while passing a varying mixture of O₂/N₂ over a thin film of RBC sample. This allows the operator to determine the oxygen saturation of Hb (SO₂) as a function of oxygen partial pressure (pO₂) (i.e., the oxygen-Hb equilibrium curve). From this curve, the oxygen partial pressure at 50% saturation (P₅₀) is determined. The BOBS was operated at 37° C. with a gas temperature offset of +1.3° C. Samples were prepared by diluting cells to a concentration of ˜70 million cells/mL using Hemox buffer (TCS Scientific Corp, New Hope, PA) with 1% additive A, and 1% additive B (TCS Scientific) at 7.4 pH. The BOBS system was refilled with 20 mL deionized water prior to each experiment session. A 2 μL sample was spread onto the glass sample plate and allowed to acclimate to system temperature before start of the run. A gas mixing system was used to automatically mix O₂ and N₂ entering the BOBS and our protocol specified an incremental increase of O₂ from 0% to 22% (incremental value of 0.1-1%), while the Soret peak intensity was measured. Data obtained from the BOBS was standardized and fit to the Hill equation in an R (RStudio, Boston, MA) script to regress the P₅₀ value and Hill coefficient (n) for cooperative oxygen binding to Hb (see A. V. Hill. The possible effects of the aggregation of the molecules of hxmoglobin on its dissociation curves. The Journal of Physiology 40 (1910) iv-vii). More specifically, the oxygen-Hb equilibrium curve was fitted to the following equation used to describe cooperative binding to ligands:

$${S}_{{O}_{2}} = \frac{{pO}_{2}^{n}}{{p}_{30}^{n} + {pO}_{3}^{n}} \qquad ( 7 )$$

Moreover, we used the Adair model to estimate the magnetic character of the samples under intermediate oxygenation states (see R. Scrima, S. Fugetto, N. Capitanio, D. L. Gatti. Hemoglobin Non-equilibrium Oxygen Dissociation Curve. BioRxiv. [Preprint.] Jan. 09, 2020. [accessed 2021 August 27]. Available from: https://doi.org/10.1101/2020.01.09.900001). Hb is a tetrameric protein where each of the four globin subunits can bind a single oxygen molecule, as presented in the following equation:

O₂+Hb(O₂)ᵢ₋₁↔Hb(O₂)ᵢ (8)
where 1≤j≤4.

Pauling determined that the magnetic moment of an unoccupied Hb subunit (i.e., not bound with oxygen) is 5.46 Bohr magnetons and when bound with oxygen is 0. Recently, it has been experimentally demonstrated that the magnetic moment of healthy RBCs is inversely proportional to oxygen partial pressure (see N. A. Smith (2019). Measurement of Red Blood Cell Oxygenation State by Magnetophoresis. [Master's Thesis, Cleveland State University]. ETD Archive. 1171. https://engagedscholarship.csuohio.edu/etdarchive/1171). In this example, we investigate the differences in the oxygen affinity among HD, TP and NTP samples, with the purpose of eventually exploiting these differences to develop a magnetophoretic device to diagnose SCD and to quantify the number or percentage of sickle RBCs in the blood of SCD patients, as well as to design a separator to isolate sickle from healthy RBCs. To calculate the magnetic moment of lib and RBCs as a function of pO₂, the Adair model was fit to oxygen-Hb equilibrium data to determine the equilibrium constants (Kᵢ), as follows:

$$\begin{matrix}{S}_{{O}_{2}} = \frac{\begin{matrix}{{K}_{1} [ {O}_{2} ] + 2 {K}_{1} {K}_{2} \{ {O}_{2} ]}^{2} + \\ 3 {K}_{1} {K}_{2} {{K}_{3} [ {O}_{2} ]}^{3} + 4 {K}_{1} {K}_{2} {K}_{3} {{K}_{4} [ {O}_{2} ]}^{4}\end{matrix}}{\begin{matrix}4 ( 1 + {{K}_{1} [ {O}_{2} ] + 2 {K}_{1} {K}_{2} \{ {O}_{2} ]}^{2} + \\ 3 {K}_{1} {K}_{2} {{K}_{3} [ {O}_{2} ]}^{3} + 4 {K}_{1} {K}_{2} {K}_{3} {{K}_{4} [ {O}_{2} ]}^{4} )\end{matrix}} & ( 9 ) \\ {K}_{i} = \frac{[ {Hb ( {O}_{2} )}_{i} ]}{[ {Hb ( {O}_{2} )}_{i - 1} ] [ {O}_{2} ]} & ( 10 )\end{matrix}$$

From the equilibrium constants Kᵢ, the mole fraction of intermediate oxygen-Hb species (Hb(O₂)ᵢ) can be quantitated (X_{Hb(O2)i}) as a function of pO₂, and this allows the determination of the magnetic moment of the samples (M_{Hb}), as follows:

$$\begin{matrix}{x}_{{Hb ( {O}_{2} )}_{i}} = \frac{{K}_{i} [ {Hb ( {O}_{2} )}_{i - 1} ] [ {O}_{2} ]}{[ Hb ] + {\sum }_{i = 1}^{4} {K}_{i} [ {Hb ( {O}_{2} )}_{i - 1} ] [ {O}_{2} ]} & ( 11 ) \\ {M}_{Hb} = 5.46 * ( 4 - ( {\sum }_{i = 1}^{4} i * {x}_{{Hb ( {O}_{2} )}_{i}} ) & ( 12 )\end{matrix}$$

### Results and Discussion

In order to understand the differences between RBCs from SCD transfused (TP samples), non-transfused patients (NTP samples) and healthy donors (BD samples), five blood samples from each source were analyzed and several RBC parameters were collected. Table 1 reports the average (and standard deviation) for each sample. From the Coulter Counter, the red cell diameter was obtained. From CTV, several RBC indices such as MCH and MCHC were estimated after measuring the settling and magnetic velocities of deoxyHb-RBCs and metHb-RBCs. Finally, the P₅₀ from BOBS is reported. These parameters and the different analyses performed are discussed in the following section below.

TABLE 1
Average and standard deviations of RBC parameters estimated from the
Coulter Counter, CTV and BOBS analyses for 15 human blood samples obtained
from healthy individuals (HD) and SCD patients requiring transfusion
therapy (TP) and not requiring transfusion therapy (NTP).

| Donor | | ρ | D | Us_{oxyHb} | Um_{oxyHb} |
|---|---|---|---|---|---|
| # | Genotype | (kg/m³) | (μm) | (μm/s) | (μm/s) |
| HD1 | HbAA | 1079 | 4.66 ± 0.43 | 1.30 ± 0.31 | −0.65 ± 0.53 |
| HD2 | HbAA | 1074 | 4.61 ± 0.43 | 1.30 ± 0.25 | −0.17 ± 0.21 |
| HD3 | HbAA | 1070 | 4.61 ± 0.45 | 1.21 ± 0.43 | −0.17 ± 0.34 |
| HD4 | HbAA | 1079 | 4.66 ± 0.45 | 1.31 ± 0.27 | −0.40 ± 0.57 |
| HD5 | HbAA | 1079 | 4.68 ± 0.42 | 1.34 ± 0.29 | −0.27 ± 0.26 |
| HD average | — | 1076 | 4.64 ± 0.44 | 1.29 ± 0.31 | −0.33 ± 0.38 |

| Donor | Us_{deoxyHb} | Um_{deoxyHb} | Us_{metHb} | Um_{metHb} | MCH_{deoxyHb} |
|---|---|---|---|---|---|
| # | (μm/s) | (μm/s) | (μm/s) | (μm/s) | (pg) |
| HD1 | 1.28 ± 0.33 | 0.77 ± 0.63 | 1.24 ± 0.34 | 0.95 ± 0.51 | 24.6 ± 16.4 |
| HD2 | 1.21 ± 0.48 | 1.07 ± 0.41 | 1.24 ± 0.39 | 1.15 ± 0.37 | 31.6 ± 12.0 |
| HD3 | 1.24 ± 0.26 | 1.38 ± 0.29 | 1.07 ± 0.46 | 1.04 ± 0.43 | 42.5 ± 11.0 |
| HD4 | 1.30 ± 0.30 | 0.86 ± 0.36 | 1.24 ± 0.33 | 0.78 ± 0.34 | 25.5 ± 10.8 |
| HD5 | 1.28 ± 0.28 | 1.21 ± 0.30 | 1.31 ± 0.34 | 1.05 ± 0.31 | 35.7 ± 10.6 |
| HD average | 1.26 ± 0.33 | 1.06 ± 0.40 | 1.22 ± 0.37 | 1.00 ± 0.39 | 32.0 ± 12.2 |

| Donor | MCHC_{deoxyHb} | MCH_{metHb} | MCHC_{metHb} | P₅₀ |
|---|---|---|---|---|
| # | (g/dL) | (pg) | (g/dL) | (mmHg) |
| HD1 | 19.0 ± 12.6 | 25.4 ± 12.9 | 20.7 ± 10.1 | 33.1 |
| HD2 | 22.6 ± 6.95 | 31.5 ± 11.8 | 22.7 ± 7.38 | 30.9 |
| HD3 | 27.8 ± 5.53 | 28.3 ± 13.1 | 21.8 ± 9.02 | 23.5 |
| HD4 | 19.3 ± 8.13 | 20.6 ± 9.93 | 16.7 ± 7.73 | 32.9 |
| HD5 | 27.3 ± 6.85 | 28.3 ± 9.50 | 21.4 ± 7.96 | 33.0 |
| HD average | 23.2 ± 8.02 | 26.8 ± 11.4 | 20.7 ± 8.43 | 30.7 |

| Donor | | ρ | D | Us_{oxyHb} | Um_{oxyHb} |
|---|---|---|---|---|---|
| # | Genotype | (kg/m³) | (μm) | (μm/s) | (μm/s) |
| NTP1 | HbSS | 1085 | 4.74 ± 0.50 | 1.24 ± 0.29 | −0.35 ± 0.52 |
| NTP2 | HbSS | 1076 | 4.73 ± 0.46 | 1.39 ± 0.32 | −0.57 ± 0.26 |
| NTP3 | HbSC | 1082 | 4.79 ± 0.45 | 1.32 ± 0.28 | −0.11 ± 0.22 |
| NTP4 | HbSB⁺ | 1066 | 4.63 ± 0.50 | 1.24 ± 0.28 | −0.30 ± 0.23 |
| NTP5 | HbSC | 1079 | 4.78 ± 0.55 | 1.28 ± 0.31 | −0.42 ± 0.29 |
| NTP average | — | 1078 | 4.73 ± 0.49 | 1.29 ± 0.29 | −0.35 ± 0.30 |

| Donor | Us_{deoxyHb} | Um_{deoxyHb} | Us_{metHb} | Um_{metHb} | MCH_{deoxyHb} |
|---|---|---|---|---|---|
| # | (μm/s) | (μm/s) | (μm/s) | (μm/s) | (μg) |
| NTP1 | 1.20 ± 0.29 | 1.18 ± 0.48 | 1.26 ± 0.36 | 0.76 ± 0.41 | 32.3 ± 14.0 |
| NTP2 | 1.29 ± 0.33 | 1.42 ± 0.38 | 1.45 ± 0.36 | 1.27 ± 0.37 | 42.4 ± 14.1 |
| NTP3 | 1.12 ± 0.36 | 1.02 ± 0.74 | 1.35 ± 0.43 | 0.84 ± 0.36 | 27.0 ± 10.1 |
| NTP4 | 1.07 ± 0.29 | 1.08 ± 0.37 | 1.25 ± 0.34 | 0.95 ± 0.40 | 32.1 ± 12.1 |
| NTP5 | 1.10 ± 0.29 | 1.01 ± 0.34 | 1.25 ± 0.33 | 0.97 ± 0.86 | 27.6 ± 10.8 |
| NTP average | 1.16 ± 0.31 | 1.14 ± 0.46 | 1.31 ± 0.36 | 0.96 ± 0.48 | 32.3 ± 12.2 |

| Donor | MCHC_{deoxyHb} | MCH_{metHb} | MCHC_{metHb} | P₅₀ |
|---|---|---|---|---|
| # | (g/dL) | (pg) | (g/dL) | (mmHg) |
| NTP1 | 31.0 ± 12.8 | 19.9 ± 10.4 | 17.9 ± 8.87 | 60.7 |
| NTP2 | 30.2 ± 8.17 | 36.4 ± 12.8 | 21.9 ± 7.09 | 40.6 |
| NTP3 | 26.9 ± 9.96 | 23.0 ± 9.76 | 17.3 ± 6.05 | 47.2 |
| NTP4 | 24.2 ± 8.20 | 27.6 ± 12.1 | 16.7 ± 7.53 | 52.0 |
| NTP5 | 26.8 ± 9.14 | 24.8 ± 11.1 | 19.3 ± 7.72 | 61.1 |
| NTP average | 27.8 ± 9.66 | 26.3 ± 11.2 | 18.6 ± 7.5 | 52.3 |

| Donor | | ρ | D | Us_{oxyHb} | Um_{oxyHb} |
|---|---|---|---|---|---|
| # | Genotype | (kg/m³) | (μm) | (μm/s) | (μm/s) |
| TP1 | HbSS | 1082 | 4.71 ± 0.51 | 1.36 ± 0.36 | −0.09 ± 0.22 |
| TP2 | HbSS | 1082 | 4.72 ± 0.52 | 1.34 ± 0.39 | −0.08 ± 0.20 |
| TP3 | HbSS | 1066 | 4.51 ± 0.44 | 1.34 ± 0.61 | −0.12 ± 0.73 |
| TP4 | HbSS | 1066 | 4.67 ± 0.52 | 1.19 ± 0.40 | −0.01 ± 0.22 |
| TP5 | HbSC | 1074 | 4.57 ± 0.46 | 1.24 ± 0.26 | −0.39 ± 0.24 |
| TP average | — | 1074 | 4.64 ± 0.49 | 1.29 ± 0.40 | −0.14 ± 0.32 |

| Donor | Us_{deoxyHb} | Um_{deoxyHb} | Us_{metHb} | Um_{metHb} | MCH_{deoxyHb} |
|---|---|---|---|---|---|
| # | (μm/s) | (μm/s) | (μm/s) | (μm/s) | (pg) |
| TP1 | 1.29 ± 0.38 | 1.50 ± 0.42 | 1.32 ± 0.35 | 1.48 ± 0.47 | 43.3 ± 14.5 |
| TP2 | 1.26 ± 0.36 | 1.58 ± 0.48 | 1.38 ± 0.42 | 1.26 ± 0.39 | 44.9 ± 16.1 |
| TP3 | 1.22 ± 0.32 | 0.99 ± 0.44 | 1.22 ± 0.40 | 1.15 ± 0.46 | 30.5 ± 10.2 |
| TP4 | 1.19 ± 0.41 | 1.02 ± 0.36 | 1.34 ± 0.47 | 1.05 ± 0.32 | 31.7 ± 13.9 |
| TP5 | 1.09 ± 0.26 | 1.19 ± 0.28 | 1.21 ± 0.30 | 0.86 ± 0.28 | 33.2 ± 9.53 |
| TP average | 1.21 ± 0.34 | 1.25 ± 0.39 | 1.29 ± 0.39 | 1.16 ± 0.38 | 36.7 ± 12.8 |

| Donor | MCHC_{deoxyHb} | MCH_{metHb} | MCHC_{metHb} | P₅₀ |
|---|---|---|---|---|
| # | (g/dL) | (pg) | (g/dL) | (mmHg) |
| TP1 | 35.8 ± 10.9 | 39.0 ± 13.0 | 30.7 ± 10.9 | 31.4 |
| TP2 | 37.6 ± 11.0 | 34.0 ± 12.6 | 24.8 ± 7.72 | 24.9 |
| TP3 | 19.7 ± 7.73 | 33.4 ± 12.1 | 20.5 ± 6.97 | 32.2 |
| TP4 | 21.9 ± 11.1 | 31.4 ± 10.4 | 17.1 ± 4.67 | 27.6 |
| TP5 | 29.6 ± 7.40 | 22.7 ± 8.32 | 17.6 ± 7.38 | 31.9 |
| TP average | 28.9 ± 9.63 | 32.1 ± 11.3 | 22.1 ± 7.52 | 29.6 |

### CTV Analysis

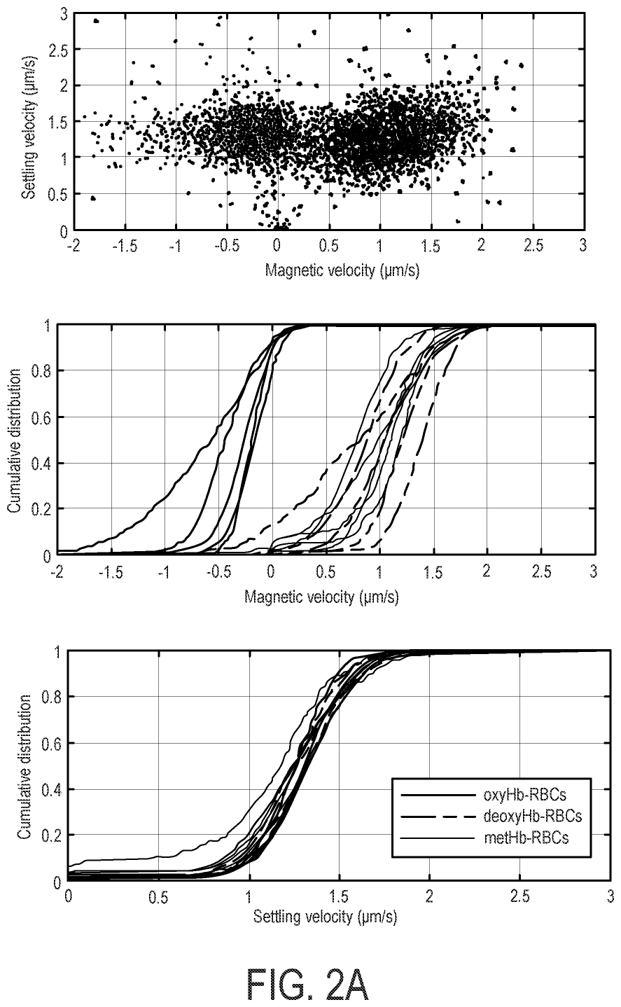

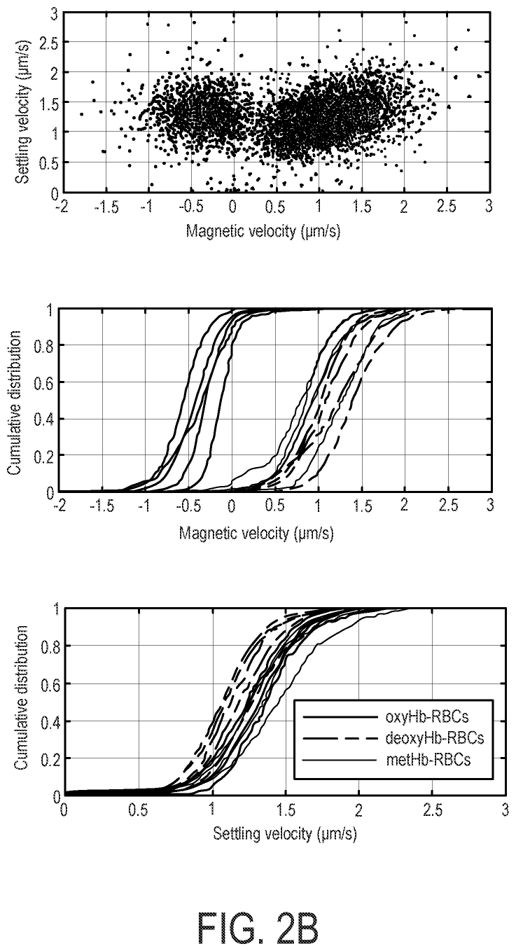

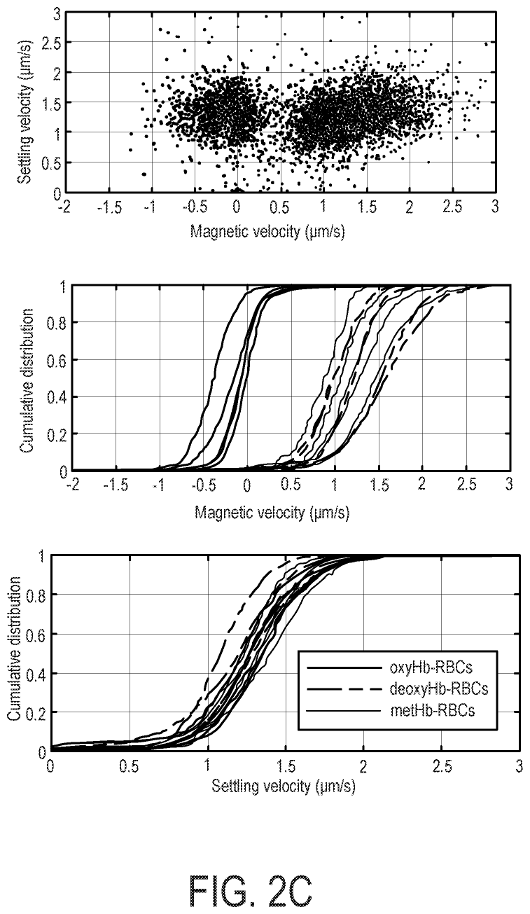

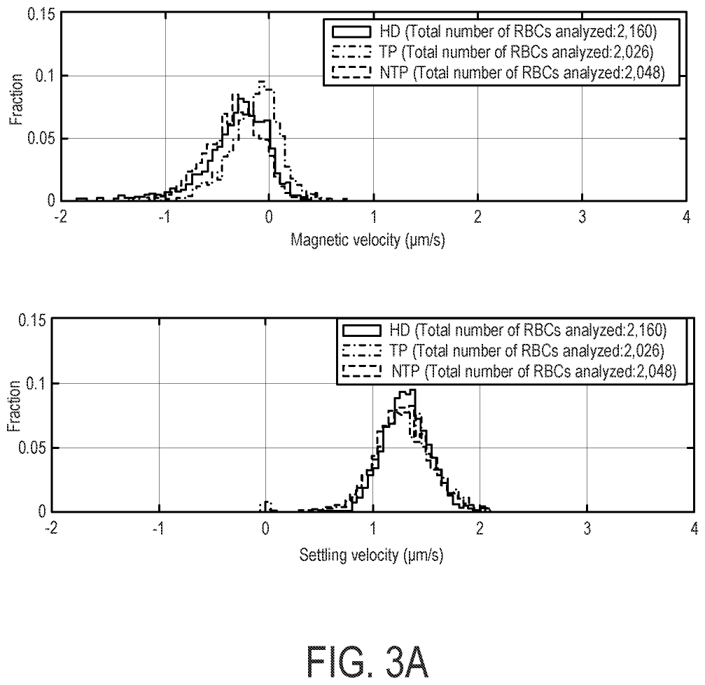

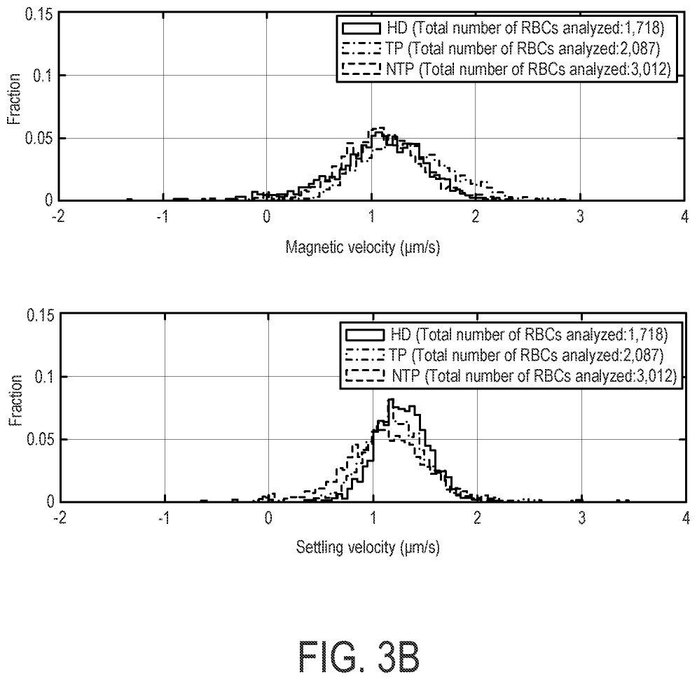

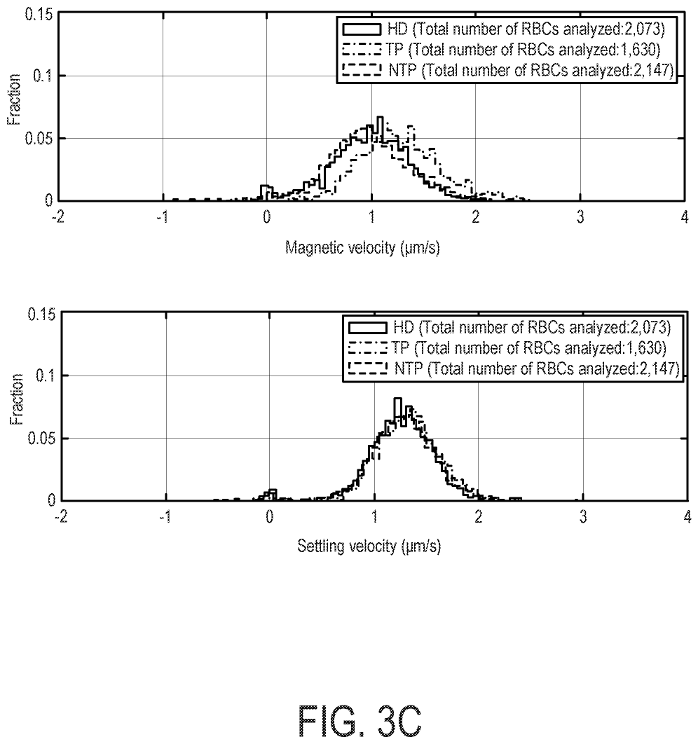

The raw data from the CTV instrument is the settling and magnetic velocities, from which the magnetic susceptibility of RBCs is determined and the MCH and MCHC RBC indices calculated. Table 1 reports the average values obtained from this analysis and FIGS. 2A-2C and 3A-3C present the CTV trajectories (cell's velocities), grouped by donor type, ND, NTP, TP, respectively. FIGS. 2A-2C present the data in form of dot plots of settling versus magnetic velocity, and the cumulative distribution curves as function of the specific velocity. FIGS. 3A-3C presents the data for each of the three states in the form of histograms, the donor samples within each of these states averaged.

The average uₘ of oxyHb-RBCs is negative for all groups. This has been previously reported and is expected since oxyHb RBCs are not only diamagnetic, but the average magnitude of this property is less than the suspending buffer (PBS). In contrast, both the deoxyHb-RBCs and metHb-RBCs have positive uₘ values, consistent with the paramagnetic property of deoxyHb and metHb. The uₘ of deoxyHb-RBCs is slightly greater than metHb-RBCs, consistent with the small difference in mobility between these chemical states of Hb, as previously reported in the literature.

Inspection of the histograms in FIGS. 3A-3C indicated a noticeable “right shift” in the magnetic mobility of the NTP donor blood relative to the HD, and TP samples. This is detectable in each of the three states, oxy, deoxy, and met. Further inspection suggests that, when comparing the settling velocity, deoxyHb-RBCs have a decreased uₛ in TP (FIG. 3C) and NTP (FIG. 3B) samples. This suggests a decreased density, decreased size/volume, or increased drag due to a change in shape when the RBCs are treated with sodium dithionite compared to the same samples treated with sodium nitrite (metHb-RBCs). However, u s data suggest that the density, size and drag are unchanged in healthy FIG. 3A. However, care should be taken since the standard deviations are sufficiently high that these observations are only speculations.

The decrease in the settling velocity of deoxyHb-RBCs obtained from SCD patients can be also observed in FIGS. 3A-3C, where the comparison between the groups of samples is performed for each chemical treatment. Especially, FIG. 3B reports a decreased uₛ of deoxyHb-RBCs for the TP and NTP samples in comparison to HD. When comparing uₘ for the three different groups (FIGS. 3A-3C), it can be seen that SCD samples (especially the TP) report the highest values, as presented also in Table 1. In fact, the largest difference in uₘ between HD and SCD samples is found when comparing oxyHb-RBCs from HD and TP. As seen in Table 1, the average oxyHb-RBC uₘ values for RD and TP are −0.33 and −0.14 μm/s, respectively. The high magnetic velocity of oxyHb-RBCs from TP samples suggests that the RBCs from these patients have impaired oxygen binding capabilities (i.e. they are not fully oxygenated). It may also suggest the effect of a slightly higher magnetization of the Fe in the oxygenated HbS molecule as compared to that in the HbA molecule, as reported by quantum-mechanical simulations. As seen in FIG. 3A, oxygenated TP samples also have a significant fraction of cells with a uₘ above 0.

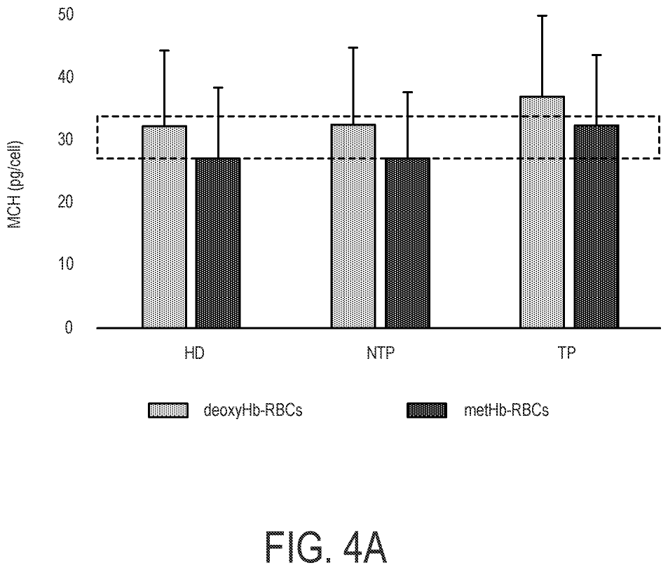

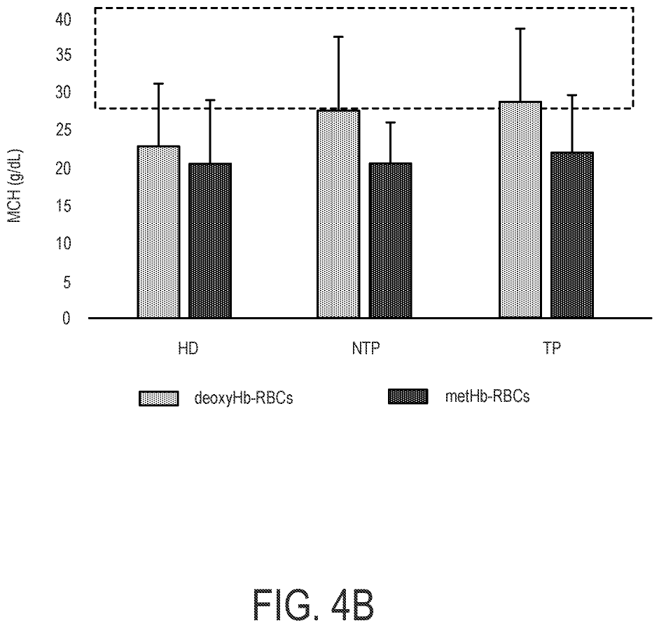

FIGS. 4A-4B report the estimated MCH and MCHC values for HD, NTP and TP samples when using the CTV data obtained from deoxyHb-RBCs and metHb-RBCs. First, it can be seen that for both parameters, the values obtained when using deoxyHb-RBCs are higher than that of metHb-RBCs. This suggests the possible formation of hemichrome when converting oxyHb into metHb. This may be due to over-oxidation with sodium nitrite, which further oxidizes metHb into hemichrome, where the Fe atom no longer has five free electrons, but instead forms a covalent bond with a neighboring histidine ring and thus, results in a diamagnetic form of Hb. DeoxyHb, on the other hand, oxidizes into metHb if treated with excess sodium dithionite. Because the susceptibilities of the two Hb forms are paramagnetic and similar in magnitude, the possibility of side reactions is ignored for deoxyHb-RBCs.

Second, the average MCH and MCHC for HD samples are lower than that of SCD samples. This is speculated to be attributed to the different intracellular Fe content, cell size/volume, shape and density of sickle RBCs. It has been suggested in the literature that dehydrated, hyperdense RBCs with high MCHC values are a distinguishing feature of SCD. These cells are believed to play an important role in the pathogenesis of the disease, due to their increased propensity to undergo polymerization and sickling. However, the fraction of hyperchromic RBCs present in the blood of SCD patients may vary according to clinical conditions, especially before or during acute painful crises.

Third, it can be observed from the plots (where the normal range for MCH and MCHC is represented by a dashed rectangle) that although the MCH values are close to the normal levels (≈30 pg/cell) for the three groups of samples, the MCHC values are below the normal range (see C. Brugnara. Reticulocyte Cellular Indices: A New Approach in the Diagnosis of Anemias and Monitoring of Erythropoietic Function. Critical Reviews in Clinical Laboratory Sciences 37 (2000) 93-130). This is attributed to the difference in estimating the RBC volume or mean corpuscular volume (MCV) using CTV data. We have previously published that even though the MCV obtained from CTV and the Coulter Counter are comparable for healthy RBCs, these values are lower than the normal MCV reference range for healthy donors (80-100 fL).

### RBC Size Distribution

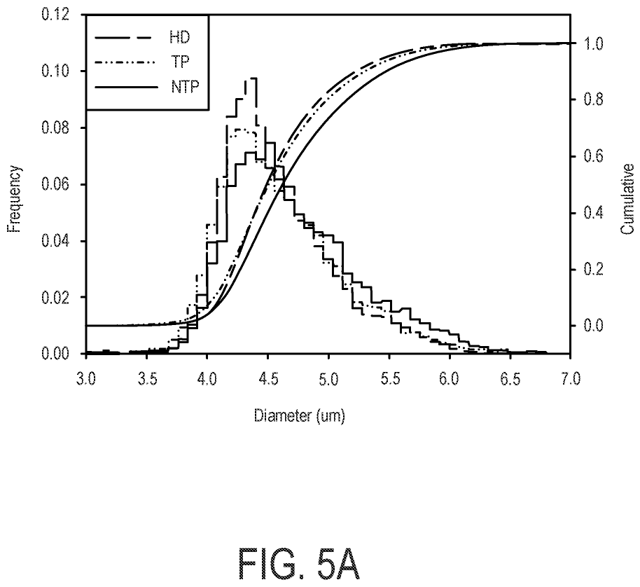

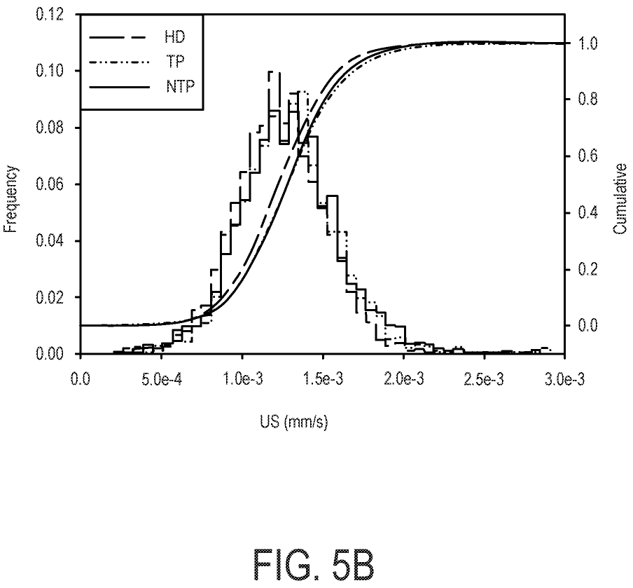

Table 1 reports the average RBC size for each individual sample and FIG. 5A presents a histogram representing the cell diameter for the combined HD, TP and NTP sample types, measured by the Coulter Counter. It can be seen from Table 1 that the average RBC sizes for both healthy donors and SCD patients are similar. Surprisingly, the average diameter for HD and TP is identical (4.64±0.44 and 4.64±0.49 μm, respectively), which is slightly less than that of SCD patients who did not receive a transfusion (4.73±0.49 μm). The larger standard deviations for TP and NTP samples are equal further demonstrated by the tall peak of the HD histogram when overlaying the RBC diameter distributions for the combined three different donor types presented in FIG. 5A. The peak diameters are very close but the NTP curve is right-shifted. The fraction of NTP RBCs with diameters greater than 5 μm is higher than that of TP and HD. This suggests that a presence of larger erythrocytes exist among those with SCD and that transfusion of smaller, healthy RBCs that are more monodisperse dilute these large cells after exchange transfusion.

FIG. 5B presents histograms of uₛ data for metHb-RBCs for the three types of donors. In this case, a small right-shift is observed for TP samples compared to HD and a further right-shift is observed for NTP samples compared to TP. The significance between these findings can be explained with Equation 2. While the size histogram suggests the presence of large cells in NTP, studying the sedimentation curve suggests that size, density, drag, or a combination of all three, are responsible for the difference. In fact, the mean density between samples suggests that NTP samples have the highest density, as seen in Table 1.

### BOBS Results

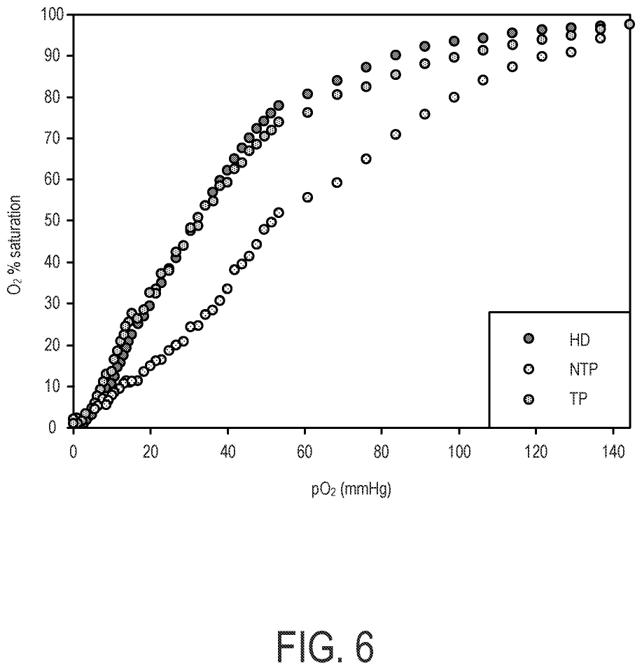

Since a “right shift” in an oxygen binding curve plot is a well-known characteristic of SCD, and as presented above, and previously published, the degree to which a RBC has bound oxygen significantly affects the magnetic susceptibility of RBCs, we used a BOBS to analyze the ability of sickle RBCs to bind oxygen as well as to estimate the magnetic properties of SCD patients' blood under intermediate oxygen levels. FIG. 6 presents overlaid oxygen equilibrium curves that plot the % saturation of O₂ in the erythrocyte Hb as a function of pO₂ for HD, TP and NTP. The data show that the P₅₀ of HD and TP RBCs are quite similar (see Table 1), with a slight lower P₅₀ for HD than TP, while the P₅₀ and overall oxygen affinity for NTP are higher and lower, respectively, compared to HD RBCs. This result with the TP donor samples could be attributed to the mixture of healthy and SCD RBCs contained in the apheresis product sample (TP), in comparison to the only native, HbS-containing RBCs of NTP samples. In fact, Table 1 reveals that the P₅₀ of HD and TP RBCs are very close and are within the healthy range (30-32 mmHg). It is noted that the BOBS software fits the raw data to the Hill equation and then reports the P₅₀ value based on the fit.

The shape of the NTP samples curve suggests decreased cooperativity compared to the other samples. O₂ saturation increases linearly with pO₂ below 20 mmHg, rather than a sigmoidal relationship, where O₂ rapidly binds to Hb due to cooperative changes in structure when Hb enters the R state. It has been reported that NTP samples have higher amounts of HbS as well as higher HbF compared to HD and TP samples. The two species have opposing effects on oxygen affinity; more HbS increases the concentration of α₂β^{S}₂ tetramers and therefore Hb in the T state, which are sensitive to polymerization. However, increased HbF yields more benign α₂β^{S}γ and α₂γ₂ tetramers upon dimer dissociation and re-association, resulting in less sickling. Additionally, high amounts of HbF result in larger cell volume, further decreasing MCHC and increasing the “delay time” of sickling, contributing to sickling reduction. Although native HbF is removed during RBC exchange apheresis, it appears that SCD patients benefit from higher oxygen affinity due to diminished HbS after receiving an exchange transfusion.

In many cases for TP and NTP samples, overlaid raw and fitted data reveal that the Hill fit overestimates Hb O₂ saturation, particularly in the 0-20 mmHg region of the oxygen equilibrium curve. This results in a small underestimation of P₅₀ and a severe overestimation of cooperativity (n). Therefore, the Adair model is chosen to describe oxygen binding equilibria moving forward, and also to estimate the magnetic moment of the samples under intermediate pO₂ values.

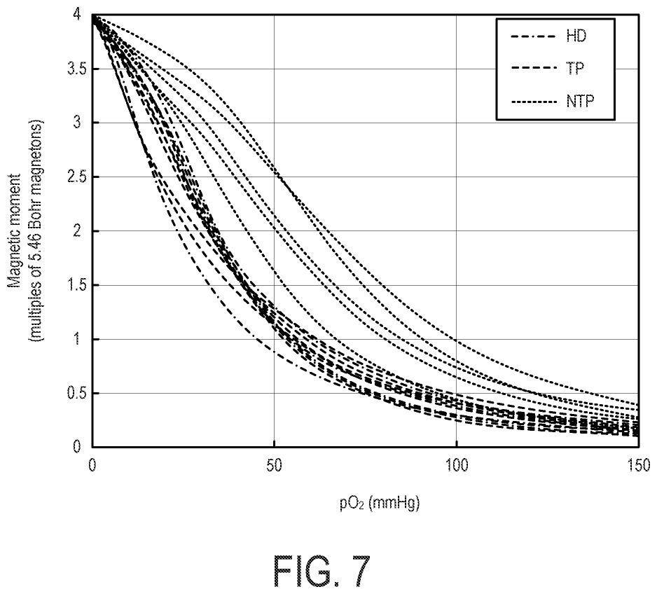

Combining oxygen saturation data and the Adair model parameters allows us to calculate the magnetic character of the samples, after determining the equilibrium constants Kᵢ for intracellular Hb bound to “i” number of oxygen atoms (where 1≤i≤4). The results are presented in FIG. 7, where the magnetic moment of the samples as a function of pO₂ is presented for the five HD, NTP and TP samples. The data reveal large changes in the Hb's magnetic moment of the RBCs (as much as a factor of 2.5) between HD and NTP samples at intermediate pO₂. This is attributed to the low O₂% saturation of TP samples under pO₂ values of 20-100 mmHg, as seen in FIG. 6. The slightly higher magnetic moment of the NTP samples oxygenated by the room air (at pO₂=150 mmHg) as compared to that seen in the HD and NTP samples is also consistent with the reported differences between O₂-HbS and O₂-HbA Fe magnetizations by quantum-mechanical simulations.

In a similar approach to the application with respect to analyzing single RBC from healthy and SCD patients, Di Caprio et al. (see High-throughput assessment of hemoglobin polymer in single red blood cells from sickle cell patients under controlled oxygen tension. PNAS, vol. 116, no. 50, pp. 25236-25242, 2019) have also reported on a microfluidic device that determines the oxygen saturation of each RBC using spectroscopic measurements (commentary to magnetic measurements discussed here). They report that not only is there a difference in oxygen saturation between SCD and normal RBCs, but that this difference is more apparent, and bimodal in partial saturation conditions. Since only healthy and transfusion discarded blood (TP in this application) was used, it is not known whether this bimodal distribution of the oxygen saturation data is the result of this mixed blood sample (healthy and SCD RBCs), or if a bimodal distribution is apparent in pure SCD

While clinical metrics such as MCH and MCHC are extremely useful in diagnosis and treatment, the small differences in average Hb between donors and modest variation within a donor require extremely high resolution and selectivity in detection/separation technology. Exploiting this additional condition, pO₂, on a heterogeneous mixture of healthy and HbS-containing cells can potentially lead to higher accuracy and efficiency in detecting and separating sickle RBCs than simply targeting the quantity of Hb alone. Furthermore, the scatter plots in FIGS. 3A and 3B suggest that cells with higher uₘ (analogous to MCH) also have higher u s (analogous to size, assuming density and drag are constant) under fully deoxygenated conditions. This leads to an unfortunate fact; the larger cells that are prone to sickling and vaso-occlusion could be misidentified as healthy cells with abundant Hb. At an intermediate pO₂, the large, Hb-rich RBCs with low concentrations of HbS that sits in the upper-right quadrant of the scatter plots in FIGS. 3A and 3B will shift to the left, due to higher oxygen affinity and therefore lower magnetic moment/susceptibility.

### Conclusion

In this example, we have described the magnetic characterization of SCD RBC samples, aiming at exploring the use of magnetophoresis for the diagnosis, quantification of sickle RBCs in the blood of SCD patients and to develop a magnetic device to separate sickle from healthy RBCs. Such a device could be employed for the separation of abnormal and unhealthy RBCs from healthy or exogenous RBCs for patients receiving a RBC exchange, which in turn would reduce the number of RBC units required for the procedure and avoid adverse effects in the transfused patients. However, in order to evaluate the technical feasibility of magnetophoresis to identify and potentially separate sickle RBCs from blood, information about the characteristics of the entire population of RBCs from SCD patients at the single cell level is required. Thus, we compared the properties of RBCs collected from SCD patients requiring and not requiring transfusion therapy with RBCs collected from healthy donors. For performing this analysis, several instruments were employed.

First, we used a Coulter Counter size/concentration analyzer, which reported similar size distributions between HD and SCD samples. However, for SCD samples, a small sub-population of cells with sizes and densities higher than that of HD was observed, to confirming the presence of dense, irreversibly sickled RBCs in SCD patients. Second, we used a cell tracking velocimetry device to quantify the response of the cells when subjected to magnetic and gravitational fields under fully oxygenation, fully deoxygenated and fully oxidized conditions. The velocity of the cells inside the device was measured for the diamagnetic state of Hb (oxyHb-RBCs) and paramagnetic states (deoxyHb-RBCs and metHb-RBCs). Our results reported that, when comparing the three Hb chemical states, deoxyHb-RBCs presented the highest magnetic velocity, followed by metHb-RBCs, for all the samples. Interestingly, when comparing the settling velocity between the different chemical treatments on the same RBC samples, SCD deoxyHb-RBCs reported the lowest value, suggesting decreased density, decreased size/volume, or increased drag due to a change in shape when the RBCs collected from SCD patients are treated with sodium dithionite compared to untreated samples or samples treated with sodium nitrite (metHb-RBCs). However, this was not observed for HD samples. When comparing the magnetic character for the three different types of samples, SCD samples, and especially TP samples, had the highest magnetic velocity, and oxyHb-RBCs reported the highest difference in um between HD and TP. The high magnetic velocity of oxyHb-RBCs from TP samples is attributed to the impaired oxygen binding capabilities of sickle RBCs (i.e., they are not fully oxygenated when in contact with O₂). RBC indices such as MCH and MCHC were also estimated from the measured uₘ and uₛ values, and the average MCH and MCHC for HD samples were lower than that of SCD samples, suggesting the presence of dehydrated hyperdense RBCs with high MCHC values in the SCD samples.

Finally, we used a blood oxygen binding system to obtain the oxygen-Hb equilibrium curves to estimate the magnetic character of SCD samples under intermediate oxygen levels. Our results confirm a difference in the ability of sickle RBCs to bind oxygen, especially for NTP, showing a much higher P₅₀ value and much lower oxygen saturation percentage in comparison to TP and HD. Moreover, the Adair model was employed to calculate the magnetic moment of samples at intermediate pO₂ values and the results for NTP samples reported the highest magnetic moment. In fact, the magnetic moment oiNTP samples were as much as twice the value obtained for TP and HD samples at intermediate pO₂. This could be attributed to the mixture of healthy and native RBCs contained in the apheresis product sample (TP), in comparison to the only native and HbS-containing RBCs of NTP samples.

### Method of Measuring Magnetically Induced Velocity of Magnetic RBCs using Cell Tracking Velocirnetry (CTV)

### Governing Equations

The magnetic force on an RBC, from which a magnetically-induced velocity of the RBC is created in a suspending fluid, is represented by:

$${F}_{mag} = ( {χ}_{RBC} - {χ}_{fluid} ) {V}_{RBC} \frac{d}{dx} [ \frac{{B}_{0}^{2}}{2 {μ}_{0}} ] = ( Δχ ) {V}_{RBC} ( \frac{1}{2 {μ}_{0}} ) ∇ {B}_{0}^{2} = ( Δχ ) {V}_{RBC} {S}_{m} \qquad ( 1 )$$

where H and B₀ are the magnetic field strength and flux density of the source, χ_{RBC} and χ_{f} are the magnetic susceptibilities of the dispersed phase (RBC) and continuous phase (suspending fluid), and V_{RBC} is the volume of the RBC. If one assumes Stokes flow (see un, J., Moore, L., Xie, Wei, Kim, J., Zborowski, M., Chalmers, J.J . (2018) Correlation of simulation/finite element analysis to the separation of intrinsically magnetic spores and red blood cells using a microfluidic magnetic deposition system. Biotechnol Bioeng 115, 1288-1300), the magnetically induced velocity is given by:

$${u}_{m} = \frac{( {χ}_{RBS} - {χ}_{f} ) {V}_{RBC}}{3 π {D}_{RBC} η {f}_{d}} {S}_{m} \qquad ( 2 )$$

where D_{RBC} is the hydrodynamic diameter of the cell, η is the viscosity of the suspending fluid, and f_{d} is a shape-dependent drag coefficient. These derivations apply to the magnetic field gradient in one dimension (see Jin, X., Yazer, M. H., Chalmers, J. J., and. Zborowski, M. (2011) Quantification of changes in oxygen release from red blood cells as a function of age based on magnetic susceptibility measurements. Analyst 136, 2996-3003).

An analogous relationship applies to the settling velocity, uₛ, of a cell or particle:

$${u}_{s} = \frac{( {ρ}_{RBS} - {ρ}_{f} ) {V}_{RBC}}{3 π {D}_{RBC} η {f}_{d}} g \qquad ( 3 )$$

where g is the gravitation acceleration (the sedimentation driving force), and ρ_{RBC} and ρ_{f} are the mass densities of the RBC and suspending fluid, respectively. Assuming that the suspending fluid is a dilute electrolyte solution in water so that ρ_{f}=ρ_{H2O} and χ_{f}=χ_{H2O}, and dividing equation 2 by 3, one obtains:

$$\frac{{u}_{m}}{{u}_{s}} = \frac{( {χ}_{RBC} - {χ}_{{H}_{2} O} )}{( {ρ}_{RBC} - {ρ}_{{H}_{2} O} )} \frac{{S}_{m}}{g} \qquad ( 4 )$$

Note that the ratio of uₘ/uₛ is independent of the size (diameter) of the cell; it is only a function of the cell density and the magnetic susceptibility.

Rearranging Equation 4, the magnetic susceptibility of each RBC is only a function of the experimentally measured ratio, uₘ/uₛ, and the density difference, Δρ, between the cell and the suspending fluid:

$${χ}_{RBC} = ( \frac{{u}_{m}}{{u}_{s}} ) ( Δρ ) ( \frac{g}{{S}_{m}} ) + {χ}_{{H}_{2} O} \qquad ( 5 )$$

### RBC Indices with CTV Measurements

We have experimentally demonstrated on normal donor blood samples that CTV can measure clinically used RBC indices such as mean corpuscular volume (MCV), mean corpuscular Hb concentration (MCHC) and mean corpuscular Hb (MCH), which were established to characterize the RBCs of anemic subjects. For example, MCHC and MCH can be calculated from:

$${MCHC}_{CTV} = \frac{( \frac{{u}_{m}}{{u}_{s}} ) ( Δρ ) ( \frac{g}{{S}_{m}} )}{{S}_{m} * ( {χ}_{m , metHb} + {χ}_{m , globin} - {χ}_{{H}_{2} O} ) * {V}_{m , Hb}} * {MW}_{Hb} \qquad ( 6 )$$

$${MCH}_{CTV} = \frac{9 * {2}^{0.5} π}{{S}_{m} * ( {χ}_{m , metHb} + {χ}_{m , globin} - {χ}_{{H}_{2} O} ) * {V}_{m , Hb}} * [ \frac{( 1.23 * {u}_{m} ) * {( 1.23 * {u}_{s} )}^{0.5} * {η}^{1.5}}{{Δρ}^{0.5} * {g}^{0.5}} ] * {10}^{3} * {MW}_{Hb} \qquad ( 7 )$$

where V_{m,Hb}=48.23 L/mol and is the molar volume of the 64 kDa, Hb molecule, χ_{m,globin}=−37,830×10⁻⁹ L/mol is the molar susceptibility of the globin chain, and χ_{H2O}=−12.97×10⁻⁹ L/mol is the molar susceptibility of water. The molar susceptibility of deoxyHb heme group is χ_{m,deoxyHb}=−50,890×10⁻⁹ L.mol, and that of metHb heme group is χ_{m,metHB}=−56,000×10⁻⁹ L/mol (all in CGS system of units). From these data, the percentages of RBCs with abnormal Hb concentration, such as hypochromic RBCs, which is a useful parameter for the detection of anemias, can be easily calculated (Hypo_{CTV}).

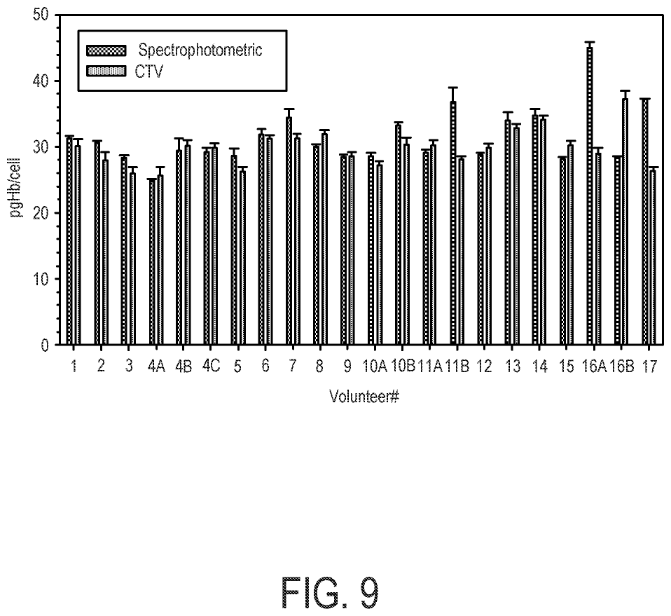

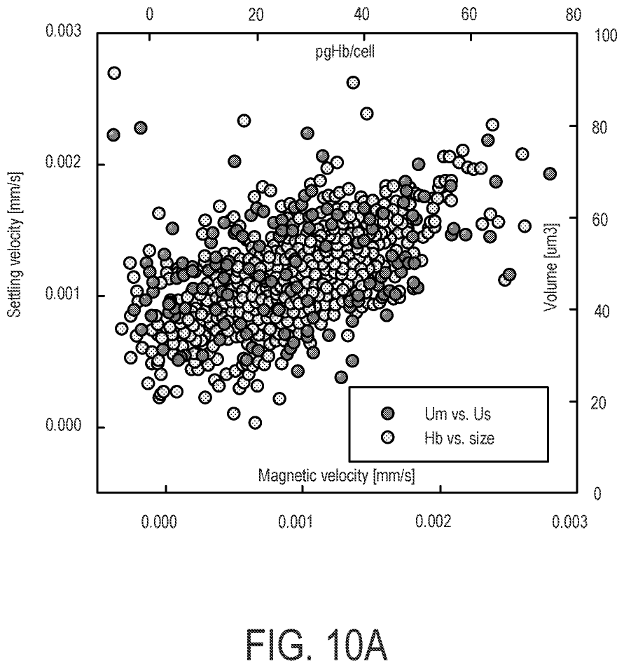

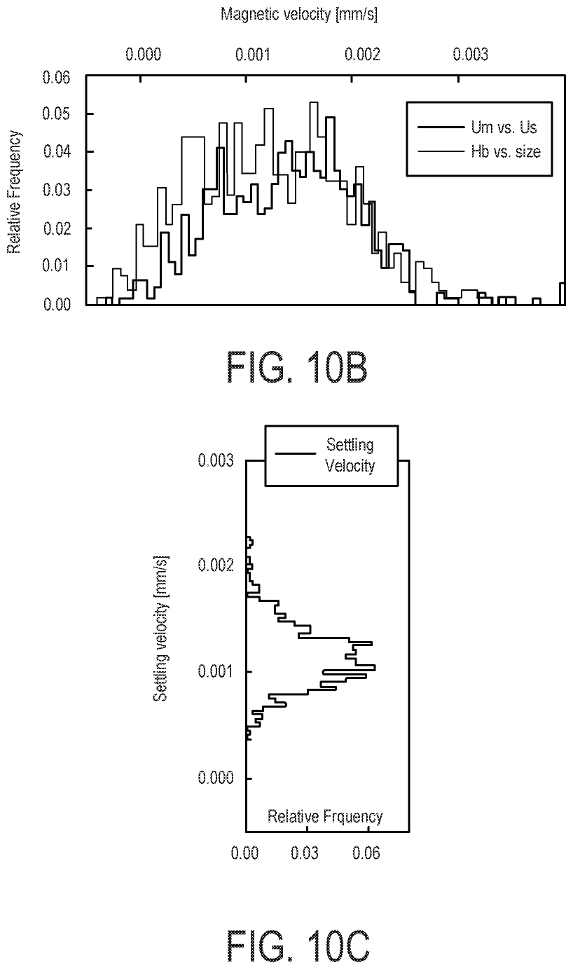

FIG. 9 shows a close correlation between the mean of the spectrophotometric and the mean of the magnetophoretic methods. However, in addition to the average values presented in FIG. 9, FIGS. 10A-10C present a representative dot plot of the data from one donor used in FIG. 9, of the settling velocity versus magnetic velocity and corresponding histograms of the magnetic field overlaid with Hb/cell and settling velocity.

### Processing of SCD Blood

1 to 2 tubes of a peripheral blood draw, or various amounts of apheresis byproduct, are processed within a couple hours. After density separation to remove platelets and white cells, the RBCs are subjected to a number of analysis techniques, including cell count and size determination with a B23005 4a Coulter Counter, cell tracking velocimetry (CTV) of both oxy-state, met-state, and deoxy-state RBCs, and oxygen saturation curves using a Blood Oxygen Binding System (BOBS) instrument. Patient information was collected and correlated to these samples, including i) prior treatments (including apheresis), ii) SCD genotype (i.e., SS, SC, etc.), pre-and post-transfusion values for iii) total Hb, iv) the breakdown of HbA, HbA2, HbF, HbS, HbC, and v) the patient disease state/pain category, from 1 to 5. The patient's most recent ferritin test was also reviewed.

### Disease State Category of Patient from which SCD Blood was Obtained

| 1 | 2 | 3 | 4 | 5 |
|---|---|---|---|---|
| Steady state | Steady state | Steady state | Steady state | In |
| with no chronic | with chronic | with no chronic | with chronic | crisis |
| pain (</=3 | pain (</=3 | pain (>3 | pain (>3 | |
| crises/year) | crises/year) | crises/year) | crises/year) | |

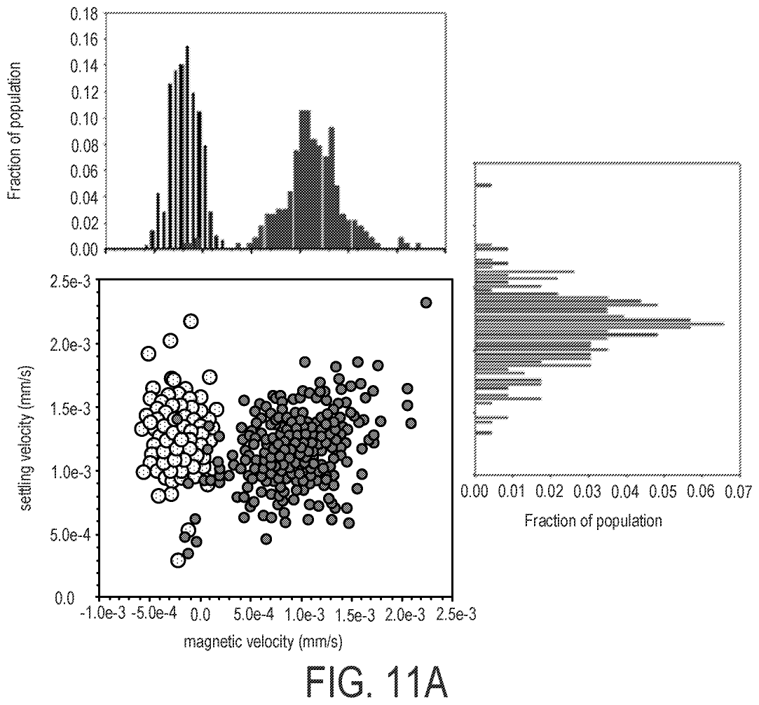

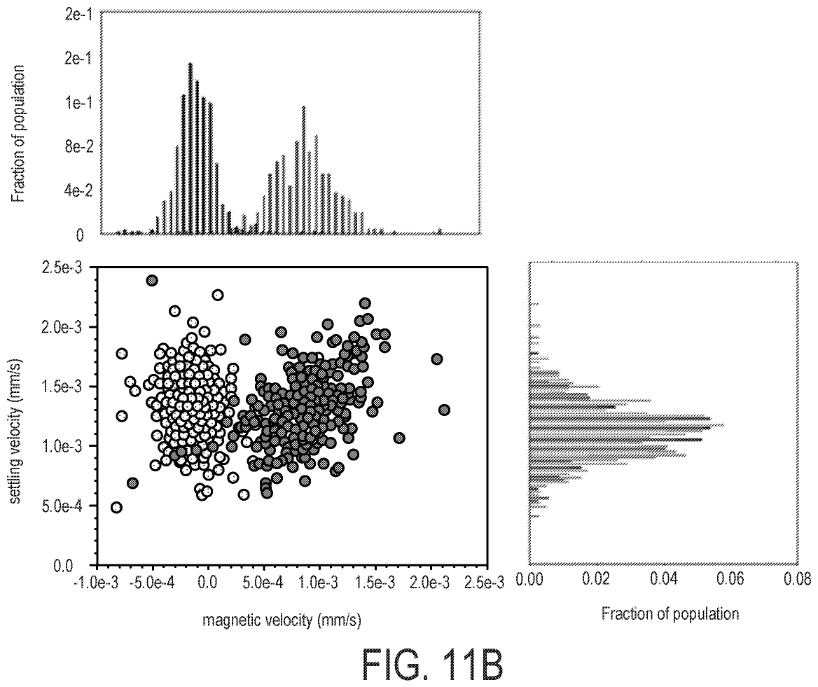

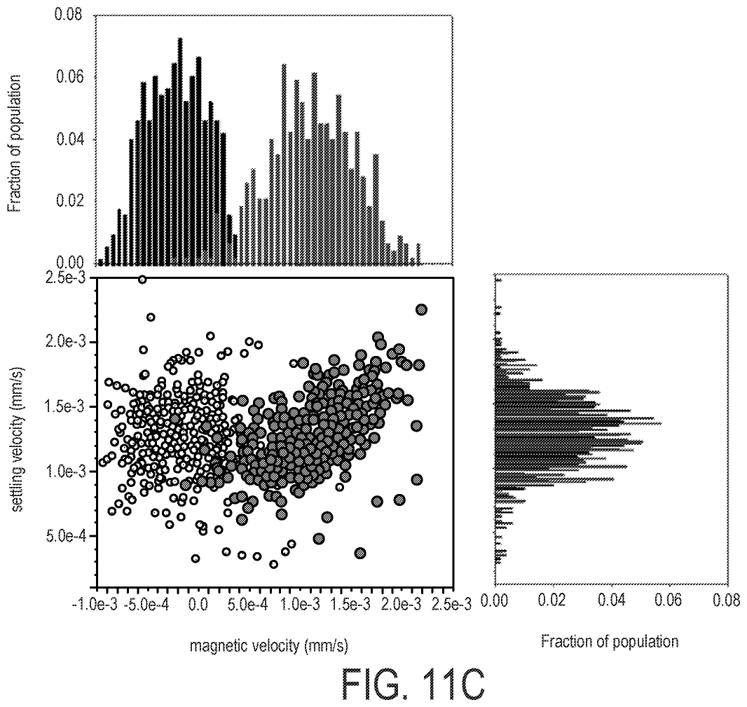

FIGS. 11A-11C are three representative results of the CTV analysis (settling and magnetic velocity) for the following types of samples: a) normal blood, b) SCD blood from a non-tranfused patient with pain category of 3, and c) SCD blood from an apheresis transfusion waste bag from a patient with pain category of 5. These figures are arranged such that the range of magnetic velocity is the same, including both the dot plot as well as histograms, for the three blood samples. First, it is noted that, in general, the relative positions of the oxyRBC and metRBC histograms are the same for all three samples, with the mean of the metRBC magnetic velocity of the pure SCD blood shifted slightly to the left. Second, the transfused patient apheresis waste sample has a significantly wider distribution in both the oxyRBC and metRBC istograms. This is expected since that specific patient started the transfusion with 30% HbS containing RBCs, and ended with 8% HbS containing RBCs. Third, the settling velocity mean and distribution does not vary much between the three types of blood. Forth, the histograms of the oxyRBCs and metRBCs overlap for the SCD blood samples, but do not for normal blood.

In addition to measuring magnetic and settling velocity of the various blood samples, we also measured the O₂ equilibrium curves of the blood using a BOBS instrument. We observed significant right shift of the curves for the pure HbS blood (indicating low oxygen affinity) and a value in between for apheresis waste blood samples.

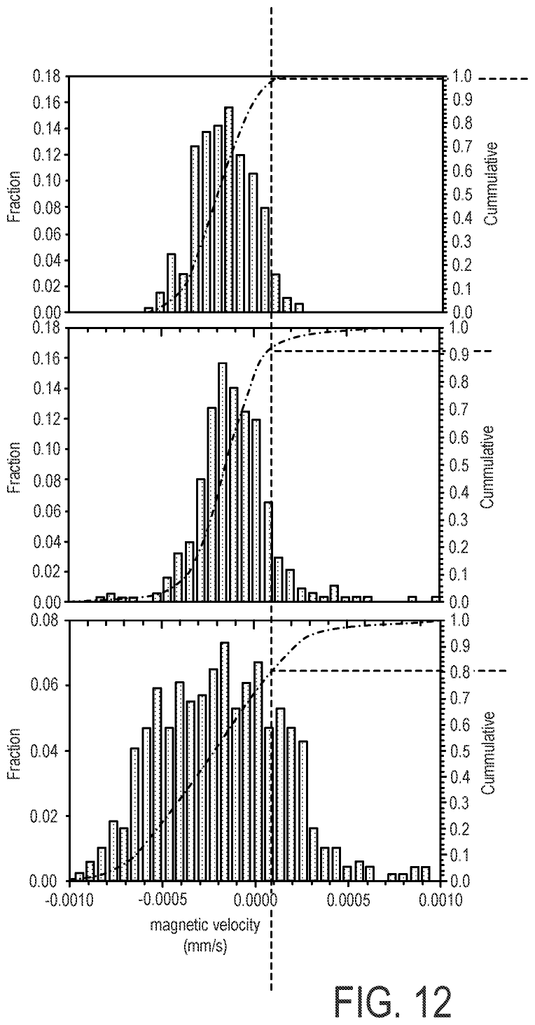

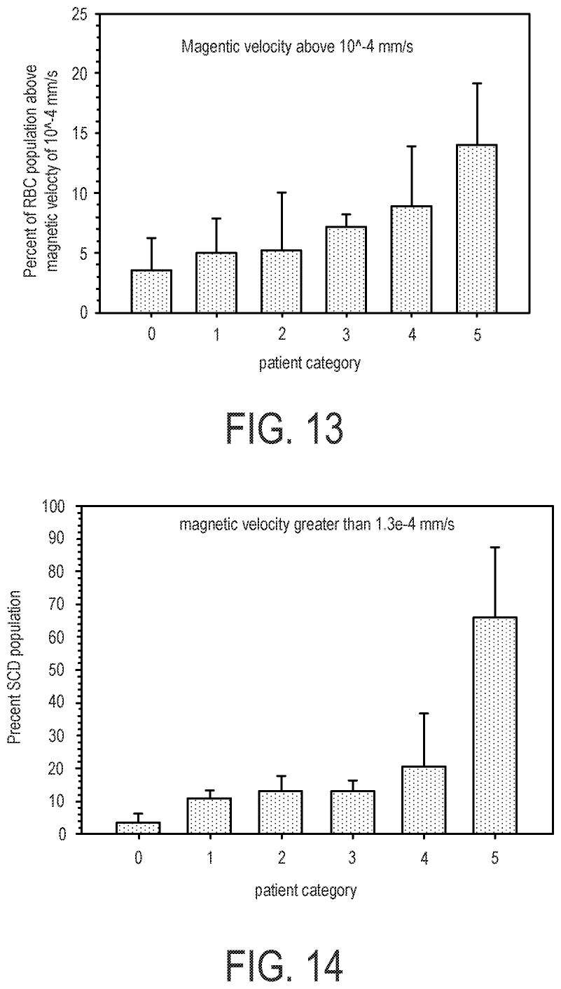

Analysis of patient samples indicates that while SCD RBCs have similar magnetic velocity compared to normal blood under total deoxygenation, we observe that not all SCD RBCs lose their magnetic velocity when exposed to room air (pO₂˜144 mmHg), which is consistent with the decreased saturation of right shifted curves at high pO₂. This observation is presented in FIGS. 12 and 13.

FIG. 12 presents the same three patient samples from FIG. 4 but with only oxyRBCs presented, and the histograms are aligned with the same x-axis. For this set of plots, a vertical dotted line is presented which represents a threshold cut off from which a percentage of RBCs with a magnetic velocity above the cutoff can be determined. To further assist in this visualization, a second y-axis is presented on the right hand side of the plots which represents the cumulative distribution of the magnetic velocity. As can be observed, there are significantly more magnetic cells in the middle (SCD, non-transfused, patient with a category 3 pain scale) and even more in the apheresis transfusion waste bag.

35 SCD patient samples were analyzed (both non-transfused clinic patients and apheresis transfusion waste), and the percentage of cells with a magnetic velocity higher than the threshold presented in FIG. 12 was determined, the results of which are found in FIG. 13. Since approximately half of the patients have the SC genotype, in contrast to the SS genotype, the data are presented representing the sum. As can be observed, there is a clear increase in the number of cells with a magnetic velocity above the threshold cut-off as the pain/disease category increases. Category 0 corresponds to normal, oxyRBCs.

A confounding factor in this analysis is the fact that all of the samples in categories 4 and 4, and half in 3, are from apheresis transfusion waste bags which is a mix of normal RBCs and SCD RBCs. Since the fraction of HbA and HbS prior to and after the transfusion was known, and the transfused blood is continuously removed and normal blood added, the following equation can be used to estimate the average percentage of SCD RBC with magnetic velocity above the threshold:

$$[ \begin{matrix}\% of pre - transfusion SCD RBCs with \\ velocity above {10}^{- 4}\end{matrix} ] = [ \frac{( \begin{matrix}fraction of cells in sample \\ analyzed with magnetic \\ velocity above threshold\end{matrix} ) - ( \begin{matrix}average fraction of normal cells with \\ magnetic velocity above \\ thresold\end{matrix} )}{fraction of HbSin sample analyzed} ] \times 100 \qquad ( 8 )$$

Using this correction factor, FIG. 14 is obtained. A significant increase in the percentage of SCD RBCs with magnetic velocity above the threshold cut-off is observed.

A number of observations can be made from FIGS. 13 and 14. First, a trend of increasing percent (fraction) of SCD RBCs with magnetic velocity of the oxy RBCs above threshold of 1×10⁻⁴ mm/s can be observed. An oxy RBC with a positive magnetic velocity is consistent with Hb tat has a reduced affinity for O₂, which is consistent with p50 data. Inspection of the histograms in FIG. 12 indicate that a small number of these RBCS have magnetic velocity that is comparable to normal RBCs fully deoxygenated. Second, if one inspects the patient in crisis, category 5, on the corrected FIG. 14, on average approximately 50% of the SCD RBCS are magnetic. Given the severity of a SCD crisis, such high numbers are not inconsistent (as all of the patient samples in category 4 and 5 are undergoing a red blood cell exchange and greater than 50% of the circulating cells are normal RBCs).

### Relationship of the Magnetic and Settling Velocity of SCD Blood as a Function of RBC Density and Correlation to Patient Pain Status

There have been reports in the literature for decades that SCD RBCs can have a greater range of densities than normal cells, and in one study by Seakens et al. 1973 (see Seakins, M. G., W. N.; Milner, P. F.; Bertles, J. F. (1973) Erythrocyte Hb-S Concentration. An Important Factor in the Low Oxygen Affinity of Blood in Sickle Cell Anemia. J. Clin. Invest. 52, 422-432), the O₂ equilibrium curves are further shifted to the right in the “heavy fraction” of SCD RBCs. Further, Kumar et al (2014) (see Ashok A. Kumara, M. R. P., Jonathan W. Hennekb, Si Yi Ryan Leeb, Gaetana D'Alesio-Spinab, Xiaoxi Yangc, and Julie Kanterd, S. S. S., Carlo Brugnarae, and George M. Whitesidesb. (2014) Density-based separation in multiphase systems provides a simple method to identify sickle cell disease. PNAS 111, 14864-14869) suggested that differences in SCD RBC density can be used as point of care detection methodology, and Thibodeaux et al. (2019) (see Thibodeauxa, S. R., Tanhehcob, Yvette C., Irwina, Leah, Jamenskya, Lita, Schella, Kevin, and O'Dohertya, U. (2019) More efficient exchange of sickle red blood cells can be achieved by exchanging the densest red blood cells: An ex vivo proof of concept study. Transfusion and Apheresis Science 58, 100-106) experimentally demonstrated that simulated SCD blood containing a mix of SCD RBCs and normal RBCs (which simulates patient blood after RBC transfusion treatment) can be separated such that one fraction has a higher dense SCD content than the other fraction. This was accomplished by manually setting the COBE Spectra apheresis instrument (Terumo BCT, Lakewood, CO) to separate at different densities than normally operated. Finally, the laboratory of Professor George Whiteside's is advocating for the use of self-assembling step-gradients in density created by aqueous multiphase systems (AMPSs) for the characterization of blood from SCD patients.

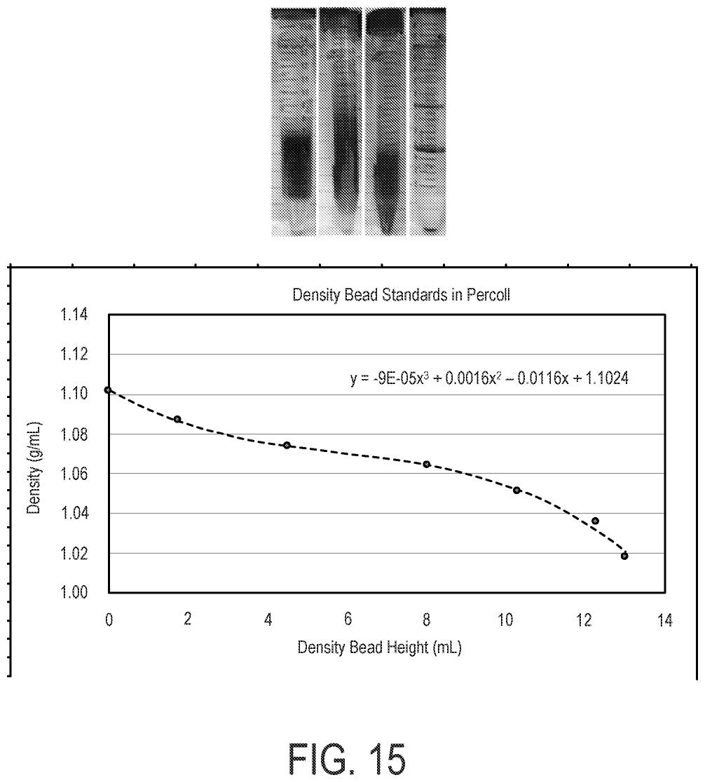

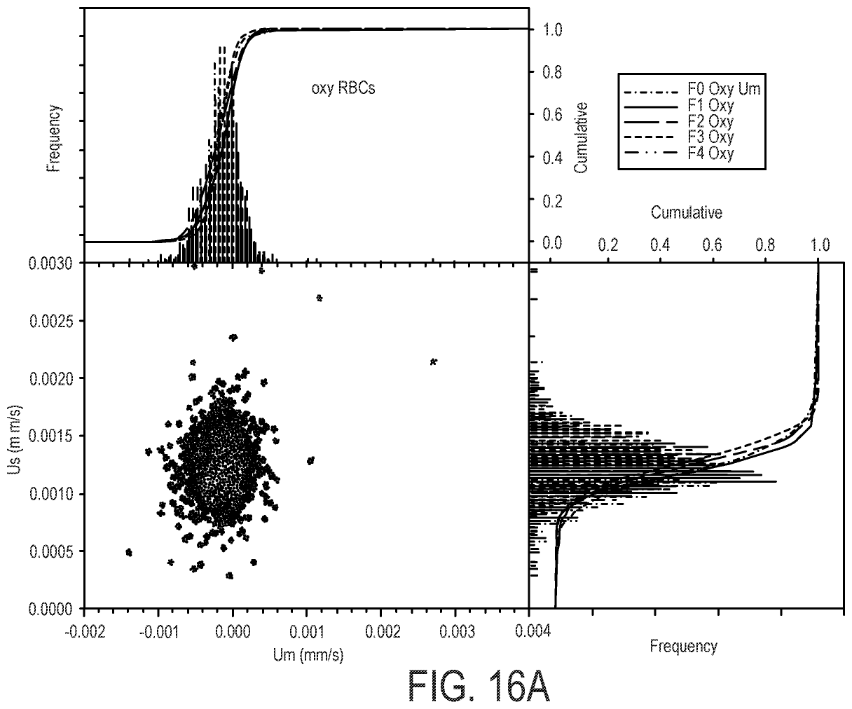

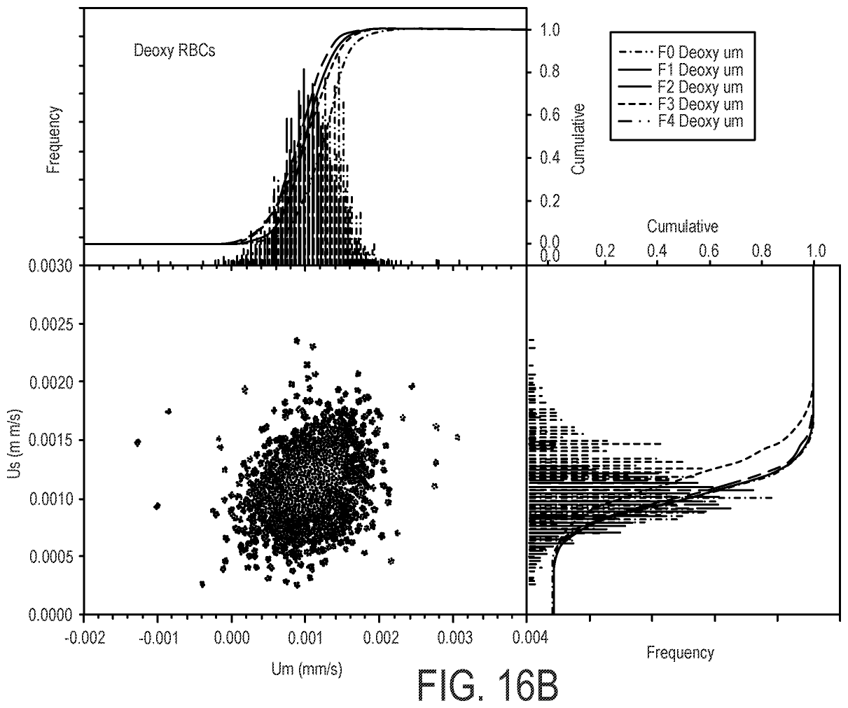

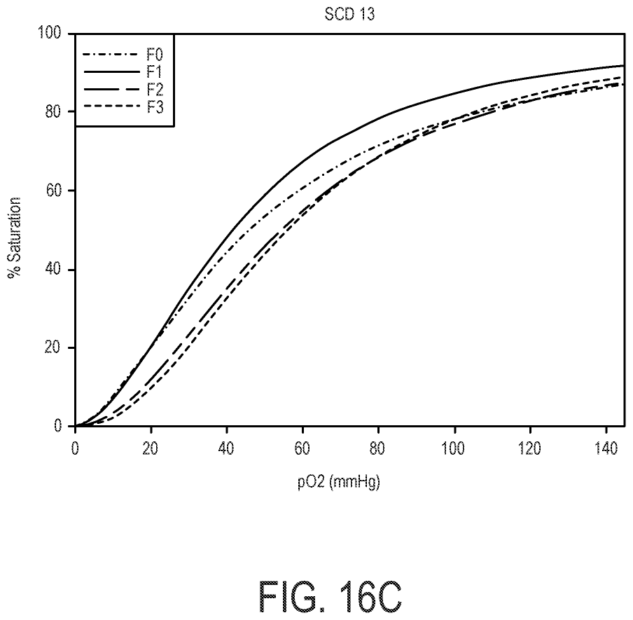

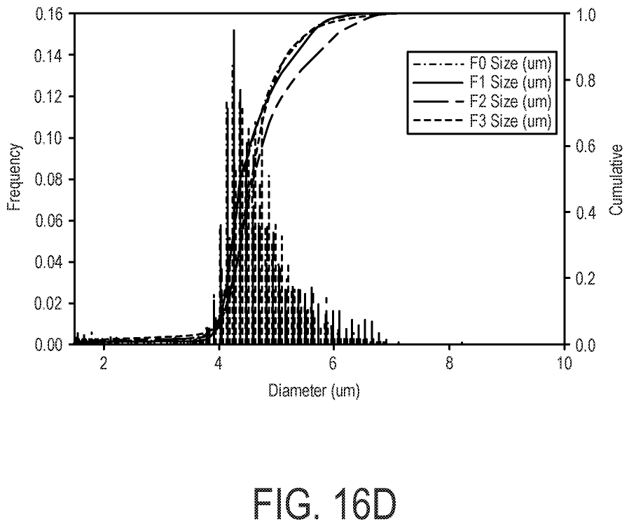

During the initial centrifugal processing to separate SCD RBCs for CTV analysis, the RCB band in the SCD blood was observed to be significantly larger (wider) than in normal blood. Percol medical was then used to further separate and quantify the density of different RBC bands. FIG. 15 presented results from a separation of normal RBCs, apheresis transfusion waste, and non-tranfused, category 1 SCD patient RBCs (left to right). SCD blood was observed to have a wider range in densities. After Percol separation, three different density aliquots were collected and further analyzed. FIGS. 16A-16D are a representation of such an analysis on non-transfused, SD patient blood that has a category pain/disease state classification. Example analysis in FIGS. 16A-16D include the settling velocity and magnetic velocity of both oxyRBCs and deoxyRBCs for each of the different density fractions, BOBs data for each of the fractions, and coulter counter measurements of the cell size for each fraction.

### O₂ Equilibrium Binding Analysis of RBCs and Conversion of RBCs into the Met/Deoxy State

To determine the O₂ affinity (P₅₀), cooperativity coefficient (n) and Adair constants of O₂-Hb binding, O₂ equilibrium curves (OECs) are measured using a Hemox Analyzer (TCS Scientific Corp., New Hope, PA) via dual-wavelength spectrophotometry and the dissolved O₂ concentration (pO₂) is measured using a Clark O₂ electrode in Hemox buffer (TCS Scientific) at 37° C. Exposure to a pO₂ of 147±1 mmHg for ˜30 minutes is used to saturate the solution with O₂. Exposure to pure N₂ for ˜30 minutes is used to deoxygenate the RBC solution. The respective absorbance of oxy-Hb and deoxy-Hb in the RBCs is used to compute the O₂ saturation (%) of the RBC solution. RBC Hb O₂-saturation is plotted as function of pO₂ to produce the OEC. To produce fully deoxygenated RBCs for CTV analysis, RBCs are deoxygenated via continuous recirculation through the liquid side of a 3M MiniModule gas/liquid exchange module (Maplewood, MN), while the gas side is fed with pure nitrogen gas (N₂). The partial pressure of O₂ in solution (pO₂) is measured using a RapidLab 248 Blood Gas Analyzer (Siemens USA, Malvern, PA). When the pO₂ is reduced below 20.0 mm Hg, sodium dithionite dissolved in N₂ purged PBS (0.1 M, pH 7.4) is then injected into the RBC solution via a needleless valve and allowed to mix until the pO₂ attains a value of 0.0 mm Hg indicative of complete deoxygenation of the RBC solution. To produce metHb containing RBCs, RBCs are incubated with sodium nitrite to oxidize Hb into metHb. Both the deoxyRBCs and metRBCs are verified via UV-visible spectroscopy. (see Weigand, M. R. H., Gómez-Pastora, J., Kim, J., Kurek, M., Hickey, R., Irwin, D. C., Buehler, P. W., Zborowski, M., Palmer. A., Chalmers, J. J. Magnetophoretic and Spectral Characterization of Oxyhemoglobin to Deoxyhemoglobin: Chemical vs Enzymatic Process)

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of identifying a test cell with a pathophysiological change associated with a change in a magnetic property as compared to a standard cell, the method comprising:
a. obtaining a test cell;
b. measuring the magnetic property of the test cell;
c. comparing the magnetic property of the test cell to the standard cell, where the standard cell is a normal cell without the pathophysiological change or a standardized version of a normal cell; and
d. identifying that the test cell has the pathophysiological change, wherein the test cell exhibits different magnetic properties as compared to the standard cell.

2. The method of claim 1, wherein the cell is selected from a monocyte, a lymphocyte, a neutrophil, an eosinophil, a basophil, a macrophage, a platelet, or an erythrocyte (red blood cell).

3-4. (canceled)

5. The method of claim 1, wherein the pathophysiological change is associated with a hemoglobinopathy.

6. The method of claim 5, wherein the hemoglobinopathy comprises sickle cell disease (SCD), sickle cell anemia, sickle cell trait, hereditary anemia, thalassemia, β-thalassemia, thalassemia major, thalassemia intermedia, α-thalassemia, or hemoglobin H disease.

7. (canceled)

8. The method of claim 5, wherein the standard cell used for comparison is from a subject without the hemoglobinopathy.

9. The method of claim 1, wherein the pathophysiological change comprises a reduced or increased level of hemoglobin (Hb), iron, or other paramagnetic atom.

10. The method of claim 1, wherein the test cell is paramagnetic.

11. (canceled)

12. The method of claim 11, wherein the magnetic property of the cell is determined using a CTV device, wherein a microscope, camera, and a magnet are used as part of the CTV device.

13. (canceled)

14. The method of claim 12, wherein the magnet comprises a permanent magnet, a superconducting magnet, or an electromagnet.

15. (canceled)

16. The method of claim 12, wherein microfluidic channels are used to track the movement of the test cell.

17. The method of claim 12, wherein the CTV device measures magnetically induced velocity (uₘ) of the cells, gravity induced settling velocity (uₛ) of the cells, or cell density, wherein the CTV device creates a magnetic energy gradient (Sₘ) which is perpendicular to gravity.

18. (canceled)

19. The method of claim 18, wherein magnetically induced horizontal and vertical velocities of the cells are measured.

20. The method of claim 19, wherein uₘ and uₛ are as follows:
$$\begin{matrix}{u}_{m} = \frac{( {χ}_{Cell} - {χ}_{Fluid} ) {V}_{Cell}}{{f}_{d} 3 π {D}_{Cell} η} {S}_{m} & ( 1 ) \\ {u}_{s} = \frac{( {ρ}_{Cell} - {ρ}_{Fluid} ) {V}_{Cell}}{{f}_{d} 3 π {D}_{Cell} η} & ( 2 )\end{matrix}$$
where the subscripts cell and fluid refer to the cell and the suspending fluid, χ is the magnetic susceptibility, ρ is the density, D and V are the diameter and volume of the cell, η is the viscosity of the suspending fluid, f_{d} is the drag coefficient, and g is the acceleration due to gravity, and where Sₘ is defined by:
$${S}_{m} = \frac{\vert ∇ {B}^{2} \vert}{2 {μ}_{0}} \qquad ( 3 )$$
where μ₀ is the permeability of free space and B is the magnetic flux density at the cell, wherein rearranging Equations (1) and (2) leads to:
$${χ}_{Cell} = {χ}_{Fluid} + \frac{{u}_{m}}{{u}_{s}} \frac{( {ρ}_{Cell} - {ρ}_{Fluid} ) g}{{S}_{m}} . \qquad ( 4 )$$

21. The method of claim 19, wherein an additive is included to modify the density of the suspending fluid, wherein the density of the suspending fluid differs from a density of a typical cell buffer.

22. (canceled)

23. The method of claim 19, wherein an additive is included to modify the magnetic susceptibility of the suspending fluid, wherein the magnetic susceptibility of the suspending fluid differs from a magnetic susceptibility of a typical cell buffer.

24-25. (canceled)

26. The method of claim 20, wherein the magnetic susceptibility of the cell is a material property of its constituents and does not depend on volume, diameter, or fluid viscosity of the cell.

27. The method of claim 26, wherein the relationship between magnetic and settling velocity and the mass and concentration of hemoglobin (Hb) in the cell, the mean corpuscular Hb (MCH) and the mean corpuscular Hb concentration (MCHC) is about:
$$MCH = \frac{9 * {2}^{0.5} π}{{S}_{m} * ( {χ}_{m , Hb} + {χ}_{m , globin} - {χ}_{{H}_{2} O} ) * {V}_{Hb}} * [ \frac{( {f}_{d} * {u}_{m} ) * {( {f}_{d} * {u}_{s} )}^{0.5} * {η}^{1.5}}{{Δρ}^{0.5} * {g}^{0.5}} ] * {10}^{3} * {MW}_{Hb} \qquad ( 5 )$$
$$MCHC = \frac{( \frac{{u}_{m}}{{u}_{s}} ) ( Δρ ) ( \frac{g}{{S}_{m}} )}{( {χ}_{m , Hb} + {χ}_{m , globin} - {χ}_{{H}_{2} O} ) ( {V}_{Hb}} * {MW}_{Hb} \qquad ( 6 )$$
where V_{Hb} is the molar volume of Hb, MW_{Hb} is the molecular weight of Hb, χ_{m,globin} is the molar susceptibility of a globin chain of Hb, and χ_{H2O} is the molar susceptibility of water.

28. The method of claim 12, wherein images of the cell's location are captured using an imaging system,
wherein the imaging system is used to calculate uₘ and uₛ, and
wherein MCH and MCHC are calculated by computer.

29-30. (calculated)

31. The method of claim 1, wherein multiple cells are analyzed in parallel.

32. The method of claim 31, wherein the cells are obtained from a human subject, an animal subject, a cell culture, or a bioreactor.

33-34. (canceled)

35. A method of diagnosing a subject with a hemoglobinopathy, the method comprising:
a. obtaining a blood sample from the subject;
b. extracting red blood cells from the blood sample;
c. measuring a magnetic property of the red blood cells;
d. comparing the magnetic property of the red blood cells to a control, wherein the control is a normal cell showing no pathophysiological change resulting from the hemoglobinopathy or a standardized version of a normal cell;
e. detecting pathological cells amongst the red blood cells, wherein said pathological cells exhibit different magnetic properties as compared to the control; and
f. diagnosing the subject with the hemoglobinopathy.

36-59. (canceled)

60. A method of treating a subject with a hemoglobinopathy, the method comprising:
a. obtaining a blood sample from the subject;
b. extracting red blood cells from the blood sample;
c. measuring a magnetic property of the red blood cells;
d. comparing the magnetic property of the red blood cells to a control, wherein the control sets a cutoff point which determined a need for a treatment;
e. determining that the subject is in need of the treatment; and
f. administering the treatment to the subject.

61-101. (canceled)
